(19) United States
(12) Patent Application Publication
(10) Pub. No.: US 2011/0097716 A1
(43) Pub. Date: Apr. 28, 2011

(54) Methods for Detecting Oligonucleotides

(75) Inventors: Francois Jean-Charles Natt, Basel (CH); Iwan Beuvink, Basel (CH); Andrew Geall, Cambridge, MA (US)

(73) Assignee: Novartis AG, Basel (CH)

(21) Appl. No.: 12/674,639

(22) PCT Filed: Aug. 21, 2009
(86) PCT No.: PCT/EP08/60948

§ 371 Date: Feb. 22, 2010

Related U.S. Application Data
(60) Provisional application No. 60/957,487, filed on Aug. 23, 2007.

(51) Int. Cl. C12Q 1/68

(52) U.S. Cl. 435/6

(57) ABSTRACT

The invention provides methods and compositions for detecting and/or quantifying nucleic acid oligonucleotides. These methods and compositions are useful for detecting and quantifying diagnostic and/or therapeutic synthetic target oligonucleotides, such as aptamers, RNAi, siRNA, antisense oligonucleotides or ribozymes in a biological sample.

### FIELD OF THE INVENTION

The invention generally relates to methods and compositions for detecting and/or quantifying modified nucleic acid oligonucleotides in a sample. These methods and compositions are useful for detecting and quantifying diagnostic and/or therapeutic synthetic modified oligonucleotides, such as aptamers, microRNA (miRNA), small interfering RNA (siRNA), and other noncoding RNA (ncRNA) molecules, antisense oligonucleotides or ribozymes in a biological sample.

### BACKGROUND OF THE INVENTION

The identification and quantitation of specific nucleic acid sequences has been an area of great interest in molecular biology over the past two to three decades. The ability to identify and to quantitate certain nucleic acids and their products has allowed the advancement of a broad range of disciplines, such as individualized medicine, including analyses of single nucleotide polymorphisms (SNPs) and evaluation of drug resistance, furthered our understanding of biochemical and molecular biological processes, and advanced cancer diagnosis and treatment, among others.

Recently much interest has focused on the newly discovered properties of certain non-coding small RNA molecules, particularly small interfering RNA (siRNA) and microRNA (miRNA) and its precursors and their effect on intracellular processes. It is currently believed that siRNA is involved in gene silencing, while miRNA is believed to be responsible for some forms of translational repression and in certain instances, gene silencing. While the interest in these small RNA molecules has risen dramatically, scientists are faced with the difficult task of identifying and quantitating these small molecules.

The siRNA molecules are double-stranded oligoribonucleotides typically 19-23 nucleotides in length. Synthetically available, they can be chemically modified and are currently developed as potential drug candidates. The pharmacological profile of such molecules is yet to be fully investigated which requires the development of novel bioanalytical methods for their detection and quantitation in a biological or clinical sample.

While much has been learned about various small RNA molecules in the past decade, much remains to be elucidated. Their small size can present problems, particularly with respect to identifying and validating candidate small RNA molecules, and detecting and quantifying known species of small RNA molecules. Conventional techniques do not adequately address these needs due to the issues of sensitivity. Accordingly, a need exists to develop more rapid, sensitive methods for detecting small RNA molecules.

### SUMMARY OF THE INVENTION

The present invention relates to compositions and methods for the rapid detection and quantification of oligonucleotide molecules such as short nucleic acids, e.g., small interfering RNAs and other short nucleic acid molecules. The invention is based in part on the discovery that the design of the reverse transcription primer comprising an oligonucleotide-binding region is highly important to the sensitivity. The improved sensitivity of the invention results in detecting a single amplified molecule. Thus, the methods and compositions of the invention provide improved, highly sensitive methods for the quantitative detection of short RNAs, for example but not limited to, oligonucleotides composed of deoxyribonucleotides and oligonucleotides composed of ribonucleotides, including without limitation, small RNA molecules, such as untranslated functional RNA, non-coding RNA (ncRNA), small non-messenger RNA (smRNA), siRNA, tRNA, tiny non-coding RNA (tncRNA), small modulatory RNA (smRNA), snoRNA, stRNA, snRNA, miRNA including without limitation miRNA precursors such as primary miRNA (pri-miRNA) and precursor miRNA (pre-miRNA), and so forth (see, e.g., Eddy, Nature Reviews Genetics 2:919-29, 2001; Storz, Science 296:1260-63, 2002; Buckingham, Horizon Symposia: Understanding the RNAissance: 1-3, 2003).

Accordingly, in one aspect, the invention pertains to a method for identifying or quantifying an oligonucleotide molecule in a sample with improved sensitivity comprising: hybridizing a reverse transcription primer to the oligonucleotide molecule, wherein the reverse transcription primer comprises an oligonucleotide molecule-binding portion having an oligonucleotide recognition sequence comprising at least 2 nucleotides at the 3′ region that are complementary to a region of the oligonucleotide molecule and an extension tail comprising at least 2 nucleotides at the 5′ region; extending the hybridized reverse transcription primer with a first extending enzyme to generate a reverse-transcribed product; hybridizing a forward primer to the reverse-transcribed product, wherein the forward primer comprises an oligonucleotide molecule-binding portion comprising at least 2 nucleotides that are the same as a region of the oligonucleotide molecule; extending the hybridized forward primer with a second extending enzyme to generate a first amplicon; hybridizing a reverse primer to the first amplicon; extending the hybridized reverse primer with the second extending enzyme to generate a second amplicon complementary to the first amplicon; detecting the amplification product; and thereby identifying or quantifying the oligonucleotide molecule. The reaction can, but need not, comprise real-time detection. In certain embodiments, an amplification step comprises multiplexing.

The method can be used to rapidly detect and quantify oligonucleotide molecules such as a small RNA molecule, a DNA molecule, a modified RNA molecule, a modified DNA molecule, an aptamer, a ribozyme, a decoy oligonucleotide, an immunostimulatory oligonucleotide. The oligonucleotides has a length comprising 10-30 nucleotides, they can be chemically modified. The oligonucleotide can also be a double stranded molecule, e.g., an siRNA. In another aspect, the present invention provides methods for the detection of an siRNA, which is capable of inhibiting at least one target gene by RNAi. The present invention is not limited to any type of siRNA or target gene or nucleotide sequence. For example, the target gene can be a cellular gene, an endogenous gene, a pathogen-associated gene, a viral gene or an oncogene.

The method can be used to directly detect and quantify oligonucleotide molecules such as a small RNA molecule, a DNA molecule, a modified RNA molecule, a modified DNA molecule, an aptamer, a ribozyme, a decoy oligonucleotide, an immunostimulatory oligonucleotide body fluids such as plasma, cerebrospinal fluid and urine without the need of RNA extraction.

Reverse transcriptase primers are disclosed that are engineered to comprise specific regions such as an SRS (siRNA-related sequence)-sequence, a probe sequence, and a reverse primer sequence. The probe sequence is positioned at a position selected from the group consisting of between the forward and reverse primer, or within the reverse transcriptase primer. First primer sets are also disclosed that include a forward primer and a corresponding reverse primer, each with an unconventionally short oligonucleotide-binding portion. The first primer and the second primer are unmodified primers. Alternatively, the first primer and the second primer are modified primers. Examples of modifications include, but are not limited to, using an LNA residue, peptide nucleic acid residue, 2′-modified RNA residue, modified nucleobases or a combination thereof.

In certain embodiments, a oligonucleotide target is combined with a reverse transcriptase primer, a first primer set, comprising a forward primer and a reverse primer, a second primer set, and an extending enzyme to form a single reaction composition. The single reaction composition is reacted under appropriate conditions and a first product, a first amplicon, an additional first amplicon, a second amplicon are generated. In certain embodiments, a first amplicon, an additional first amplicon, a second amplicon, or combinations thereof, are detected and the oligonucleotide is identified and/or quantitated. In certain embodiments, the detecting comprises an integral reporter group, a reporter probe, an intercalating agent, or combinations thereof. In certain embodiments, the amplifying, the detecting, and the quantitating comprise Q-PCR or another real time technique. Certain embodiments comprise an end-point detection technique.

In another embodiment, the hybridization occurs in two separate reaction mixtures, wherein the reverse transcriptase primer is present in a first reaction mixture and is used to generate a reverse transcribed product, and the forward and reverse primers are present in a second reaction mixture, wherein the reverse transcribed product from the first reaction mixture is used as a template for the forward and reverse primers in the second reaction mixture. In certain embodiments, the disclosed methods comprise forming at least two different reaction compositions, for example but not limited to, a first reaction composition and a second reaction composition. Some embodiments further comprise at least a third reaction composition. In certain embodiments, two primer sets per oligonucleotide target are used in three or four amplification steps that occur in at least two different reaction compositions, including without limitation, a first reaction composition and a multiplicity of different second reaction compositions, and can but need not take place in the same reaction vessel. According to such methods, the amplification steps that occur in the first reaction compositions typically include: (i) generating a first product using the reverse transcription primer, reverse primer of the first primer set, (ii) generating a first amplicon using the first product as the template and the corresponding forward primer of the first primer set, and optionally, (iii) generating additional first amplicons using additional forward and reverse primers of the corresponding first primer set. When the first stage is completed, a second reaction composition is typically formed by combining (i) all or part of the reacted first reaction composition, (ii) a second primer set, which can, but need not include universal primers, primers comprising unique hybridization tags, or both, (iii) a third extending enzyme, and optionally, (iv) a reporter probe. Under appropriate reaction conditions second amplicons are generated using the additional first amplicons as templates.

In one embodiment, the oligonucleotide molecule-binding portion of the reverse transcriptase primer comprises a nucleotide sequence that is at least 90% complementary with the oligonucleotide molecule. In another embodiment, the oligonucleotide molecule-binding portion of the reverse transcriptase primer comprises about 2-17 nucleotides that are complementary with the oligonucleotide molecule, where the oligonucleotide molecule is about 4-19 nucleotides in length. In one embodiment, the oligonucleotide molecule-binding portion of the reverse primer comprises about 2-30 nucleotides that are complementary to the region of the oligonucleotide molecule. In one embodiment, the oligonucleotide molecule-binding portion of the forward primer comprises about 2-30 nucleotides having the same sequence as the region of the oligonucleotide molecule.

The current teachings also provide reporter probes that are particularly useful in the disclosed methods. Those in the art will appreciate, however, that conventional reporter probes may also be used in the disclosed methods. In one embodiment, the step of detecting the amplification product comprises detecting the first amplicon with a first detection probe, the second amplicon with a second detection probe, and detecting both the first and second amplicons with multiple detection probes. In one embodiment, the first detection probe is a double stranded DNA intercalating agent. In one embodiment, the first detection probe is SYBR Green. In another embodiment, the first and second detection probes are signal emitting probes that binds with the oligonucleotide molecule binding portion using Watson-Crick base pairing. Examples of signal emitting probes include, but are not limited to, FAM, VIC, JOE, NED, CY5 dye, CY3-dye, TAMRA labeled probe, an MBG probe, scorpion probe and molecular beacon. In a preferred embodiment, the detection probe comprises a FAM/TAMRA detection group.

The methods of the invention unexpectedly result in an improved sensitivity that for quantifying the oligonucleotide molecule. In one embodiment, the sensitivity is improved by a factor of at least 10-100,000 fold, or at least 100-10,000 fold detected using a signal intensity readout. In another embodiment, the sensitivity for quantifying the oligonucleotide is improved to detect oligonucleotide molecules in a concentration range of about 1 molecule to about 1×10¹⁰ molecules and about 100 molecules to about 1×10⁹ molecules.

The methods and compositions of the invention can be used to detect the oligonucleotide molecule after administration of the oligonucleotide molecule into a subject by a clinically relevant route selected but from the group consisting of intratracheal, intranasal, intracerebral, intrathecal, colorectal, oral, intramuscular, intraarticular, topical including vaginal, lung delivery, intraocular, intraperitoneal, intravenous, and subcutaneous, administration. The oligonucleotide molecule can be formulated with a pharmaceutical carrier capable of facilitating delivery to and/or uptake by the target cells. Selected from, but not limited to, neutral liposomes, cationic liposomes or lipoplexes, cationic polymers or polyplexes, neutral polymers, nanoparticles, double stranded RNA binding proteins, calcium phosphate, cell penetrating peptides, viral proteins and viral particles, antibodies and empty bacterial envelopes. The sample to be tested using the methods of the invention is selected from the group consisting of a fluid, a tissue, a cell, and a tumor.

Also provided are kits that can be used to perform the disclosed methods. In certain embodiments, kits comprise a first primer set and a first extending enzyme. In certain embodiments, the disclosed kits further comprise, a second extending enzyme, a third extending enzyme, a second primer set, a reporter probe, a reporter group, a reaction vessel, or combinations thereof. These and other features of the present teachings are set forth herein.

### BRIEF DESCRIPTION OF DRAWINGS

The skilled artisan will understand that the drawings, described below, are for illustration purposes only and are not intended to limit the scope of the present teachings in any way.

FIG. 1 shows the quantification of siRNAs in plasma using two-step RT-PCR;

FIG. 2 shows the comparison of one-step RT-PCR;

FIG. 3 shows the comparison of two-step RT-PCR based detection of siRNAs ND9227 using SYBR Green I or FAM/TAMRA labeled probes as readout;

FIG. 4 shows the results from absolute quantification of siRNA in rat lung;

FIG. 5 is a schematic showing siRNA detection using FAM/TAMRA probes; and

FIG. 6 is a schematic showing the minimal sequence required for a reverse transcription primer.

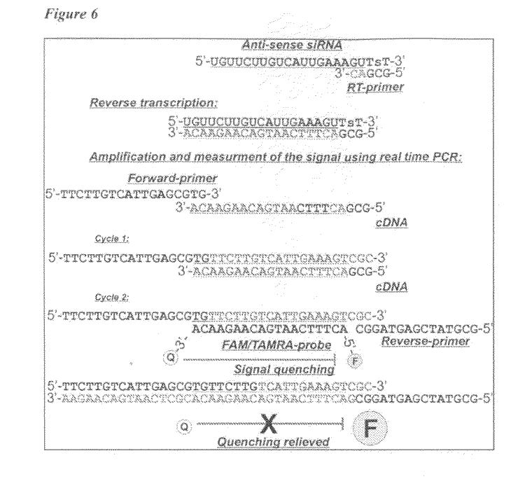

### DETAILED DESCRIPTION OF THE INVENTION

It is to be understood that both the foregoing general description and the following detailed description are exemplary and explanatory only and are not intended to limit the scope of the current teachings. In this application, the use of the singular includes the plural unless specifically stated otherwise. The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. In the event that one or more of the incorporated literature and similar materials contradicts this application, including but not limited to defined terms, term usage, described techniques, or the like, this application controls.

### I. DEFINITIONS

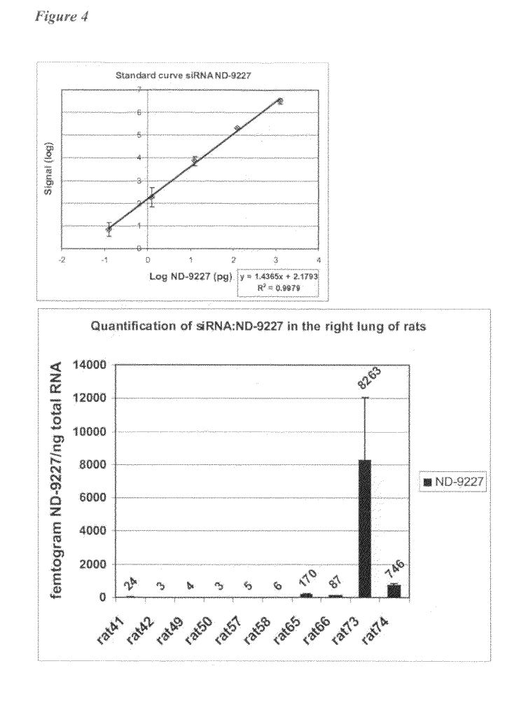

The term “short interfering nucleic acid”, “siRNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of inhibiting or down regulating gene expression or viral replication, for example by mediating RNA interference “RNAi” or gene silencing in a sequence-specific manner; see for example Zamore et al., 2000, Cell, 101, 25 33; Bass, 2001, Nature, 411, 428 429; Elbashir et al., 2001, Nature, 411, 494 498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818 1819; Volpe et al., 2002, Science, 297, 1833 1837; Jenuwein, 2002, Science, 297, 2215 2218; and Hall et al., 2002, Science, 297, 2232 2237; Hutvagner and Zamore, 2002, Science, 297, 2056 60; McManus et al., 2002, RNA, 8, 842 850; Reinhart et al., 2002, Gene & Dev., 16, 1616 1626; and Reinhart & Bartel, 2002, Science, 297, 1831). Non limiting examples of siRNA molecules of the invention are shown in FIGS. 4 6, and Tables II and III herein. For example the siRNA can be a double-stranded oligonucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siRNA is assembled from a single oligonucleotide, where the self-complementary sense and antisense regions of the siRNA are linked by means of a nucleic acid based or non-nucleic acid-based linker(s). The siRNA can be a oligonucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siRNA can be a circular single-stranded oligonucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular oligonucleotide can be processed either in vivo or in vitro to generate an active siRNA molecule capable of mediating RNAi. The siRNA can also comprise a single stranded oligonucleotide having nucleotide sequence complementary to nucleotide sequence in a target nucleic acid molecule or a portion thereof (for example, where such siRNA molecule does not require the presence within the siRNA molecule of nucleotide sequence corresponding to the target nucleic acid sequence or a portion thereof), wherein the single stranded oligonucleotide can further comprise a terminal phosphate group, such as a 5′-phosphate (see for example Martinez et al., 2002, Cell, 110, 563 574 and Schwarz et al., 2002, Molecular Cell, 10, 537 568), or 5′,3′-diphosphate. In certain embodiments, the siRNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the siRNA molecules of the invention comprise nucleotide sequence that is complementary to nucleotide sequence of a target gene. In another embodiment, the siRNA molecule of the invention interacts with nucleotide sequence of a target gene in a manner that causes inhibition of expression of the target gene. As used herein, siRNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. Applicant describes in certain embodiments short interfering nucleic acids that do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siRNA molecules that do not require the presence of ribonucleotides within the siRNA molecule to support RNAi can however have an attached linker or linkers or other attached or associated groups, moieties, or chains containing one or more nucleotides with 2′-OH groups. Optionally, siRNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.” As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules of the invention can be used to epigenetically silence genes at both the post-transcriptional level or the pre-transcriptional level. In a non-limiting example, epigenetic regulation of gene expression by siRNA molecules of the invention can result from siRNA mediated modification of chromatin structure to alter gene expression (see, for example, Verdel et al., 2004, Science, 303, 672 676; Pal-Bhadra et al., 2004, Science, 303, 669 672; Allshire, 2002, Science, 297, 1818 1819; Volpe et al., 2002, Science, 297, 1833 1837; Jenuwein, 2002, Science, 297, 2215 2218; and Hall et al., 2002, Science, 297, 2232 2237).

The term “Amplicons” is used in a broad sense herein and includes amplification products of the disclosed methods. First products (including but not limited to reverse transcribed products), first amplicons, additional first amplicons, second amplicons, or combinations thereof, fall within the intended scope of the term Amplicons.

The terms “hybridizing” and “annealing”, and variations of these terms such as annealed, hybridization, anneal, hybridizes, and so forth, are used interchangeably and mean the nucleotide base-pairing interaction of one nucleic acid with another nucleic acid that results in the formation of a duplex, triplex, or other higher-ordered structure. The primary interaction is typically nucleotide base specific, e.g., A:T, A:U and G:C, by Watson-Crick and Hoogsteen-type hydrogen bonding. In certain embodiments, base-stacking and hydrophobic interactions may also contribute to duplex stability. Conditions under which reporter probes and primers hybridize to complementary and substantially complementary target sequences are well known in the art, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the hybridizing region of the primers and reporter probes and their complementary sequences, the pH, the temperature, the presence of mono- and divalent cations, the proportion of G and C nucleotides in the hybridizing region, the viscosity of the medium, and the presence of denaturants. Such variables influence the time required for hybridization. The presence of certain nucleotide analogs or groove binders in the primer or reporter probe can also influence hybridization conditions. Thus, the preferred annealing conditions will depend upon the particular application. Such conditions, however, can be routinely determined by persons of ordinary skill in the art, without undue experimentation.

As used herein, the terms “oligonucleotide”, “polynucleotide”, “nucleic acid”, and “nucleic acid sequence” are generally used interchangeably and include single-stranded and double-stranded polymers of nucleotide monomers, including 2′-deoxyribonucleotides (DNA) and ribonucleotides (RNA) linked by inter-nucleotide phosphodiester bond linkages, or inter-nucleotide analogs, and associated counter ions, e.g., H⁺, NH₄⁺, trialkylammonium, tetraalkylammonium, Mg²⁺, Na⁺, and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. Nucleic acids typically range in size from a few monomeric units, e.g. 5-40, when they are sometimes referred to in the art as oligonucleotides, to several thousands of monomeric nucleotide units. Nucleic acid sequences are shown in the 5′ to 3′ orientation from left to right, unless otherwise apparent from the context or expressly indicated differently; and in such sequences, “A” denotes adenine, “C” denotes cytosine, “G” denotes guanine, “T” denotes thymine, and “U” denotes uracil, unless otherwise apparent from the context.

The term “nucleotide base”, as used herein, refers to a substituted or unsubstituted aromatic ring or rings. In certain embodiments, the aromatic ring or rings contain a nitrogen atom. In certain embodiments, the nucleotide base is capable of forming Watson-Crick or Hoogsteen-type hydrogen bonds with a complementary nucleotide base. Exemplary nucleotide bases and analogs thereof include, but are not limited to, naturally-occurring nucleotide bases adenine, guanine, cytosine, 5 methyl-cytosine, uracil, thymine, and analogs of the naturally occurring nucleotide bases, including without limitation, 7-deazaadenine, 7-deazaguanine, 7-deaza-8-azaguanine, 7-deaza-8-azaadenine, N6-.DELTA.2-isopentenyladenine (6iA), N6-.DELTA.2-isopentenyl-2-methylthioadenine (2 ms6iA), N2-dimethylguanine (dmG), 7-methylguanine (7 mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT Published Application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different, —R, —OR, —NR.sub.2 azide, cyanide or halogen groups, where each R is independently H, C₁-C₆ alkyl, C₂-C₇ acyl, or C₅-C₁₄ aryl. Exemplary riboses include, but are not limited to, 2′-(C₁-C₆)alkoxyribose, 2′-(C₅-C₁₄)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT Published Application Nos. WO 98/22489, WO 98/39352, and WO 99/14226; and Braasch and Corey, Chem. Biol. 8:1-7, 2001). “LNA” or “locked nucleic acid” is a DNA analogue that is conformationally locked such that the ribose ring is constrained by a methylene linkage between, for example but not limited to, the 2′-oxygen and the 3′- or 4′-carbon or a 3′-4′ LNA with a 2′-5′ backbone. The conformation restriction imposed by the linkage often increases binding affinity for complementary sequences and increases the thermal stability of such duplexes. Exemplary LNA sugar analogs within a oligonucleotide include, but are not limited to, the structures: where B is any nucleotide base.

The 2′- or 3′-position of ribose can be modified to include, without limitation, hydrogen, hydroxy, methoxy, ethoxy, allyloxy, isopropoxy, butoxy, isobutoxy, methoxyethyl, alkoxy, phenoxy, azido, cyano, amido, imido, amino, alkylamino, fluoro, chloro and bromo. Nucleotides include, but are not limited to, the natural D optical isomer, as well as the L optical isomer forms (see, e.g., Garbesi Nucl. Acids Res. 21:4159-65 (1993); Fujimori (1990) J. Amer. Chem. Soc. 112:7435; Urata, (1993) Nucleic Acids Symposium Ser. No. 29:69-70). When the nucleotide base is purine, e.g., A or G, the ribose sugar is attached to the N.sup.9-position of the nucleotide base. When the nucleotide base is pyrimidine, e.g. C, T, or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2nd Ed., Freeman, San Francisco, Calif.).

One or more of the pentose carbons of a nucleotide may be substituted with a phosphate ester having the formula: where alpha is an integer from 0 to 4. In certain embodiments, alpha is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. Reviews of nucleotide chemistry can be found in, among other places, Shabarova, Z. and Bogdanov, A. Advanced Organic Chemistry of Nucleic Acids, VCH, New York, 1994; and Blackburn and Gait.

The term “nucleotide analog”, as used herein, refers to embodiments in which the pentose sugar or the nucleotide base or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counter ions.

Also included within the definition of nucleotide analog are monomers that can be polymerized into oligonucleotide analogs in which the DNA/RNA phosphate ester or sugar phosphate ester backbone is replaced at least in part by a different type of inter-nucleotide linkage. Exemplary oligonucleotide analogs include, but are not limited to, peptide nucleic acids (PNAs), in which the sugar phosphate backbone of the oligonucleotide is replaced by a peptide backbone comprising a amide bond. It is to be understood that the term “PNA” as used herein, includes pseudocomplementary PNAs (pcPNAs) unless otherwise apparent from the context. (See, e.g., Datar and Kim, Concepts in Applied Molecular Biology, Eaton Publishing, Westborough, Mass., 2003, particularly at pages 74-75; Verma and Eckstein, Ann. Rev. Biochem. 67:99-134, 1998; Goodchild, Bioconj. Chem., 1:165-187, 1990; Braasch and Corey, Methods 23:97-107, 2001; Demidov et al., Proc. Natl. Acad. Sci. 99:5953-58, 1999).

Nucleic acids include, but are not limited to, genomic DNA, cDNA, hnRNA, mRNA, rRNA, tRNA, small RNA molecules, including without limitation, miRNA and miRNA precursors, siRNA, stRNA, snoRNA, other non-coding RNAs (ncRNA), fragmented nucleic acid, nucleic acid obtained from the nucleus, the cytoplasm, subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribooligonucleotides and 2′-deoxyribooligonucleotides according to the structural formulae below: wherein each B is independently the base moiety of a nucleotide, e.g., a purine, a 7-deazapurine, a purine or purine analog substituted with one or more substituted hydrocarbons, a pyrimidine, a pyrimidine or pyrimidine analog substituted with one or more substituted hydrocarbons, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl, (C2-C7) acyl or (C5-C14) aryl, cyanide, azide, or two adjacent Rs are taken together to form a bond such that the ribose sugar is 2′,3′-didehydroribose; and each R′ is independently hydroxyl or where alpha is zero, one or two.

In certain embodiments of the ribooligonucleotides and 2′-deoxyribooligonucleotides illustrated above, the nucleotide bases B are covalently attached to the C1′ carbon of the sugar moiety as previously described. The terms “nucleic acid”, “nucleic acid sequence”, “polynucleotide”, and “oligonucleotide” can also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains a nucleotide analog or a phosphate ester analog or a pentose sugar analog. Also included within the definition of nucleic acid analogs are nucleic acids in which the phosphate ester or sugar phosphate ester linkages are replaced with other types of linkages, such as N-(2-aminoethyl)-glycine amides and other amides (see, e.g., Nielsen et al., 1991, Science 254: 1497-1500; PCT Publication No. WO 92/20702; U.S. Pat. Nos. 5,719,262 and 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, J. Org. Chem. 52: 4202, 1987); methylene(methylimino) (see, e.g., Vasseur et al., J. Am. Chem. Soc. 114:4006, 1992); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., PCT Publication No. WO 92/20702; Nielsen, Science 254:1497-1500, 1991); and others (see, e.g., U.S. Pat. No. 5,817,781; Frier & Altman, Nucl. Acids Res. 25:4429, 1997 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate. See also, Scheit, Nucleotide Analogs, John Wiley, New York, (1980); Englisch, Agnew. Chem. Int. Ed. Engl. 30:613-29, 1991; Agarwal, Protocols for Oligonucleotides and Analogs, Humana Press, 1994; and S. Verma and F. Eckstein, Ann. Rev. Biochem. 67:99-134, 1999.

The term “reporter group” is used in a broad sense herein and refers to any identifiable tag, label, or moiety. The skilled artisan will appreciate that many different species of reporter groups can be used in the present teachings, either individually or in combination with one or more different reporter group. The term reporter group also encompasses an element of multi-element indirect reporter systems, including without limitation, affinity tags; and multi-element interacting reporter groups or reporter group pairs, such as fluorescent reporter group-quencher pairs, including without limitation, pairs comprising fluorescent quenchers and dark quenchers, also known as non-fluorescent quenchers (NFQ).

The term “threshold cycle” or “CT” is used in reference to quantitative or real-time analysis methods and indicates the fractional cycle number at which the amount of analyte, for purposes of the current teachings, Amplicons and including without limitation, one or both strands of any of these, reaches a fixed threshold or limit. Thresholds can be manually set by the user or determined by the software of a real-time instrument. Exemplary real-time instruments include, the ABI PRISM™ 7000 Sequence Detection System, the ABI PRISM™ 7700 Sequence Detection System, the ABI PRISM™ 7900HT Sequence Detection System, the ABI PRISM™ 7300 Real-Time PCR System (Applied Biosystems), the Smart Cycler System (Cepheid, distributed by Fisher Scientific), the LightCycler™ System (Roche Molecular), and the Mx4000 (Stratagene, La Jolla, Calif.). In certain embodiments, such real-time quantitation comprises reporter probes, including without limitation, conventional reporter probes and the reporter probes of the present teachings, intercalating dyes, including without limitation, FAM/TAMRA probes, ethidium bromide and SYBR Green I or its equivalent, or such reporter probes and intercalating dyes. Descriptions of real-time analysis can be found in, among other places, Essentials of Real Time PCR, Applied Biosystems P/N 105622, 2002; PCR: The Basics from background to bench, McPherson and Moller, Bios Scientific Publishers Limited, Oxford UK, 2000 (“PCR: The Basics”), particularly at Section 3.3; Real-Time PCR: An Essential Guide, Edwards et al., eds., Horizon Bioscience, Norwich, UK; and Handbook of Fluorescent Probes and Research Products, 9.sup.th ed., R. Haugland, Molecular Probes, Inc., 2002 (“Molecular Probes Handbook”), particularly at Section 8.3.

The term “first product” refers to the nucleotide sequence that results when the reverse primer of the first primer set, hybridized to the second region of the corresponding target nucleotide, is extended by an extending enzyme in a primer extension reaction. When the target oligonucleotide is an RNA molecule, for example but not limited to, a small RNA molecule, the first product can be referred to as a reverse-transcribed product. Those in the art will appreciate that the generation of first products according to the current teachings are at least similar to generating reverse transcripts in conventional RT-PCR techniques.

As used herein, the term “oligonucleotide-binding portion” refers to the sequence of a forward primer that is the same as the first region of the corresponding target or that sequence of a reverse primer that is complementary to the second region of the corresponding target. Those in the art will appreciate that when the target is a polynucleotide, the term “oligonucleotide-binding portion” is interchangeable with the term oligonucleotide-binding portion and when the target is a small RNA molecule, the term “small RNA molecule-binding portion” is interchangeable with the term oligonucleotide-binding portion. Thus, the terms oligonucleotide-binding portion, oligonucleotide binding portion, and small RNA molecule-binding portion are used in reference to target sequences in general, oligonucleotide targets, and small RNA molecule targets, respectively. The term “primer-binding portion” refers to that sequence of the forward or reverse primers of a first primer set to which the corresponding primers of the second primer set specifically hybridize. Typically, the primers of the second primer set are employed to enable the first product, the first amplicon, the additional first amplicon, or combinations thereof, to be amplified, including without limitation techniques comprising multiple amplification cycles such as PCR. In certain embodiments, a primer of a second primer set is utilized to amplify the corresponding first product, a strand of a corresponding first amplicon, a strand of the corresponding additional first amplicon, a strand of a corresponding second amplicon, or combinations thereof.

The terms “universal base” or “universal nucleotide” are generally used interchangeably herein and refer to a nucleotide analog (including nucleoside analogs) that can substitute for more than one of the natural nucleotides or natural bases in oligonucleotides. Universal bases typically contain an aromatic ring moiety that may or may not contain nitrogen atoms and generally use aromatic ring stacking to stabilize a duplex. In certain embodiments, a universal base may be covalently attached to the C-1′ carbon of a pentose sugar to make a universal nucleotide. In certain embodiments, a universal base does not hydrogen bond specifically with another nucleotide base. In certain embodiments, a nucleotide base may interact with adjacent nucleotide bases on the same nucleic acid strand by hydrophobic stacking. Universal nucleotides and universal bases include, but are not limited to, deoxy-7-azaindole triphosphate (d7AITP), deoxyisocarbostyril triphosphate (dICSTP), deoxypropynylisocarbostyril triphosphate (dPICSTP), deoxymethyl-7-azaindole triphosphate (dM7AITP), deoxylmPy triphosphate (dImPyTP), deoxyPP triphosphate (dPPTP), deoxypropynyl-7-azaindole triphosphate (dP7AITP), 3-methyl isocarbostyril (MICS), 5-methyl isocarbyl (5MICS), imidazole4-carboxamide, 3-nitropyrrole, 5-nitroindole, hypoxanthine, inosine, deoxyinosine, 5-fluorodeoxyuridine, 4-nitrobenzimidazole, and PNA-bases, including pcPNA bases. Descriptions of universal bases can be found in, among other places, Loakes, Nucl. Acids Res. 29:2437-47, 2001; Berger et al., Nucl. Acids Res. 28:2911-14, 2000; Loakes et al., J. Mol. Biol. 270:426-35, 1997; Verma and Eckstein, Ann. Rev. Biochem. 67:99-134, 1998; Published. PCT Application No. US02/33619, and U.S. Pat. Nos. 6,433,134 and 6,433,134.

The terms “oligonucleotide target”, “target oligonucleotide”, or “target” refers to the nucleic acid sequence whose identity, presence, absence, and/or quantity is being evaluated using the methods and kits of the present teachings. In certain embodiments, the target sequence comprises a oligonucleotide, which may or may not comprise a deoxyribonucleotide, or an RNA molecule such as a miRNA precursor, including without limitation, a pri-miRNA, a pre-miRNA, or a pri-miRNA and a pre-miRNA. In some embodiments, the oligonucleotide target comprises a small RNA molecule, including without limitation, a miRNA, a siRNA, a stRNA, a snoRNA, other ncRNA, and the like.

The term “reporter probe” refers to a sequence of nucleotides, nucleotide analogs, or nucleotides and nucleotide analogs, that binds to or anneals with an Amplicon, and when detected, including but not limited to a change in intensity or of emitted wavelength, is used to identify and/or quantify the corresponding target oligonucleotide. Most reporter probes can be categorized based on their mode of action, for example but not limited to: nuclease probes, including without limitation TaqMan™ probes (see, e.g., Livak, Genetic Analysis: Biomolecular Engineering 14:143-149, 1999; Yeung et al., BioTechniques 36:266-75, 2004); extension probes such as scorpion primers, Lux™ primers, Amplifluors, and the like; hybridization probes such as molecular beacons, Eclipse probes, light-up probes, pairs of singly-labeled reporter probes, hybridization probe pairs, and the like; or combinations thereof. In certain embodiments, reporter probes comprise an amide bond, an LNA, a universal base, or combinations thereof, and include stem-loop and stem-less reporter probe configurations. Certain reporter probes are singly-labeled, while other reporter probes are doubly-labeled. Dual probe systems that comprise FRET between adjacently hybridized probes are within the intended scope of the term reporter probe.

In certain embodiments, a reporter probe comprises a fluorescent reporter group, a quencher reporter group (including without limitation dark quenchers and fluorescent quenchers), an affinity tag, a hybridization tag, a hybridization tag complement, or combinations thereof. In certain embodiments, a reporter probe comprising a hybridization tag complement anneals with the corresponding hybridization tag, a member of a multi-component reporter group binds to a reporter probe comprising the corresponding member of the multi-component reporter group, or combinations thereof. Exemplary reporter probes include TAM/FAMRA probes, TaqMan probes; Scorpion probes (also referred to as scorpion primers); Lux™ primers; FRET primers; Eclipse probes; molecular beacons, including but not limited to FRET-based molecular beacons, multicolor molecular beacons, aptamer beacons, PNA beacons, and antibody beacons; reporter group-labeled PNA clamps, reporter group-labeled PNA openers, reporter group-labeled LNA probes, and probes comprising nanocrystals, metallic nanoparticles and similar hybrid probes (see, e.g., Dubertret et al., Nature Biotech. 19:365-70, 2001; Zelphati et al., BioTechniques 28:304-15, 2000). In certain embodiments, reporter probe detection comprises fluorescence polarization detection (see, e.g., Simeonov and Nikiforov, Nucl. Acids Res. 30:e91, 2002).

In addition to such conventional reporter probes, the reporter probes of the current teachings, can be used in the detection, identification, and quantitation of corresponding target oligonucleotides. The reporter probes of the current teachings include gap probes, certain chimeric probes, and gap probes that comprise chimeric sequences. Gap probes are designed to specifically hybridize with sequences in Amplicons that are the counterpart of the gap sequences of small RNA molecules, i.e., that sequence in a small RNA molecule that is not the same sequence as the oligonucleotide-binding portion of the corresponding forward primer nor is it complementary to the oligonucleotide-binding portion of the corresponding reverse primer, but are located between these sequences. The reporter probes include: (i) homopolymer probes and also (ii) heteropolymer or chimeric probes. Exemplary homopolymer probes of the current teachings include without limitation, DNA probes, RNA probes, LNA probes, 2′ O-alkyl nucleotide probes, phosphoroamidite probes (for example but not limited to, N3′-P5′ phosphoroamidite probes and morpholino phosphoroamidite probes), 2′-fluoro-arabino nucleic acid (FANA) probes, cyclohexene nucleic acid (CeNA) probes, tricycle-DNA (tcDNA) probes, and PNA probes (see, e.g., Kurreck, Eur. J. Biochem., 270:1628-44, 2003). The chimeric probes, include without limitation, DNA-PNA chimeric probes, DNA-LNA chimeric probes, DNA-2′ O-alkyl chimeric probes, and so forth. In certain embodiments, such DNA chimeric probes comprise at least two deoxyribonucleotides that are usually located at the 5′-end of the probe, but not always.

The reporter probes further comprise a reporter group, and in certain embodiments, comprise a fluorescent reporter group-quencher pair. In certain embodiments, reporter probes are designed to hybridize only with the gap sequences or the complement of gap sequences found in Amplicons. Those in the art will appreciate that even in the presence of “primer dimer” artifacts, which sometimes accompany certain amplification techniques and which may contain some sequences in common with the target oligonucleotide, only bona fide Amplicons will contain gap sequences or their complement and thus can stably hybridize with the disclosed reporter probes that hybridize only to the gap (assuming appropriate stringency conditions which those in the art understand can be calculated using various well-known algorithms or determined empirically). In certain embodiments, the Amplicon-binding portion of a reporter probe is designed to hybridize with the gap sequences or the gap sequence complements found in Amplicons and also to a few nucleotides adjacent to the gap sequences, typically one or two additional nucleotides on one or both sides of the Amplicon gap sequences.

In certain embodiments, chimeric reporter probes are disclosed that comprise a reporter group, two or more deoxyribonucleotides, and downstream, a multiplicity of nucleotide analogs. Typically such nucleotide analogs are selected because they do not readily serve as templates for DNA polymerases or reverse transcriptases and thus are not amplified during primer extension reactions. Exemplary non-extendable nucleotide analogs include without limitation, locked nucleic acids (LNAs), peptide nucleic acids (PNAs), and 2′ O-alkyl nucleotides, for example but not limited to, 2′ O-methyl nucleotides and 2′ O-ethyl nucleotides. In certain embodiments, chimeric reporter probes comprise a reporter group and at least two deoxyribonucleotides located upstream from at least four PNAs. In certain embodiments, a chimeric reporter probe comprises a fluorescent reporter group-quencher pair. In certain embodiments, a fluorescent reporter group is located upstream from at least two deoxyribonucleotides or is attached to at least one of the two deoxyribonucleotides, and the quencher is located downstream (or vice versa) to form a fluorescent reporter group-quencher pair, which may or may not comprise fluorescence resonance energy transfer (FRET). Those in the art will appreciate that such reporter probes can be particularly useful for certain detection techniques, such as nuclease assays, including without limitation, TaqMan.RTM. assays.

The disclosed first primer sets include forward primers and reverse primers, each comprising unusually short oligonucleotide-binding portions, i.e., forward primers with no more than 2 nucleotides that have the same sequence as the first target region and reverse primers with no more than 2 nucleotides that are complementary to the second target region. In certain embodiments, the oligonucleotide-binding portion of the forward primers contain 2, 3, 4, 5, 6, or 7 nucleotides that have the same sequence as the corresponding first region of the target. In certain embodiments, the oligonucleotide-binding portion of the reverse primers contain 2, 3, 4, 5, 6, or 7 nucleotides that are complementary to the corresponding second region of the target. In certain embodiments, the forward primers and the reverse primers further comprise an additional portion that is upstream from the oligonucleotide-binding portion and can, but need not be, a primer-binding portion. When present, such primer-binding portions are designed to selectively hybridize with the respective primers of the corresponding second primer set. Thus, when incorporated in Amplicons, additional amplification is possible using the corresponding second primer set and an appropriate extending enzyme.

The second primer sets of the current teachings comprise a first primer and a second primer that are designed to anneal to regions of Amplicons that correspond to the primer-binding portions of the forward and reverse primers, respectively, of the corresponding first primer set. In certain embodiments, a primer of a second primer set is a universal primer. In certain embodiments, a second primer set comprises a universal forward primer and a universal reverse primer. In certain embodiments, a primer of a second primer set further comprises a hybridization tag, an affinity tag, a reporter group, or combinations thereof. In certain embodiments, a hybridization tag allows the corresponding Amplicon to be identified. In certain embodiments, a first primer of the second primer set comprises a first universal priming sequence and the second primer of the corresponding second primer set comprises a second universal priming sequence. In certain embodiments, one primer of a second primer set comprises a universal priming sequence and the other primer of the corresponding second primer set comprises a hybridization tag, including without limitation, a unique hybridization tag that can be used to subsequently identify the corresponding Amplicon.

The binding portions of the first primer set primers, the second primer set primers, and the reporter probes of the current teachings are of sufficient length to permit specific annealing to complementary regions of corresponding target sequences, corresponding Amplicons. The criteria for designing sequence-specific nucleic acid primers and reporter probes are well known to those in the art. Detailed descriptions of nucleic acid primer and reporter probe design can be found in, among other places, Diffenbach and Dveksler, PCR Primer, A Laboratory Manual, Cold Spring Harbor Press (1995); R. Rapley, The Nucleic Acid Protocols Handbook (2000), Humana Press, Totowa, N.J. (“Rapley”); Schena; and Kwok et al., Nucl. Acid Res. 18:999-1005 (1990). Primer and reporter probe design software programs are also commercially available, including without limitation, Primer Express, Applied Biosystems; Primer Premier and Beacon Designer software, PREMIER Biosoft International, Palo Alto, Calif.; Primer Designer 4, Sci-Ed Software, Durham, N.C.; Primer Detective, ClonTech, Palo Alto, Calif.; Lasergene, DNASTAR, Inc., Madison, Wis.; Oligo software, National Biosciences, Inc., Plymouth, Minn.; iOligo, Caesar Software, Portsmouth, N.H.; and RTPrimerDB on the world wide web at realtimeprimerdatabase.ht.st or at medgen31.urgent.be/primerdatabase/index (see also, Pattyn et al., Nucl. Acid Res. 31:122-23, 2003).

Those in the art understand that primers and reporter probes suitable for use with the disclosed methods and kits can be identified empirically using the current teachings and routine methods known in the art, without undue experimentation. For example, suitable primers, primer sets, and reporter probes can be obtained by selecting candidate target oligonucleotides from the relevant scientific literature, including but not limited to, appropriate databases and using computational algorithms (see, e.g., miRNA Registry, on the world-wide web at sanger-ac.uk/Software/Rfam/miRNA/index; MiRscan, available on the web at genes/mit.edu/mirscan; miRseeker; and Carter et al., Nucl. Acids Res. 29(19):3928-38, 2001). When oligonucleotides of interest are identified, test primers and/or reporter probes can be synthesized using well known synthesis techniques and their suitability can be evaluated in the disclosed methods and kits (see, e.g., Current Protocols in Nucleic Acid Chemistry, Beaucage et al., eds., John Wiley & Sons, New York, N.Y., including updates through August 2004 (“Beaucage et al.”); Blackburn and Gait; Glen Research 2002 Catalog, Sterling, Va.; The Glen Report 16(2):5, 2003, Glen Research; Synthetic Medicinal Chemistry 2003/2004, Berry and Associates, Dexter, Mich.; and PNA Chemistry for the Expedite™ 8900 Nucleic Acid Synthesis System User's Guide, Applied Biosystem). Those in the art will appreciate that the melting temperature (Tm) of a primer or reporter probe can be increased by, among other things, incorporating a minor groove binder, substituting a an appropriate nucleotide analog for a nucleotide (i.e., a chimeric probe), or using a homopolymer probe comprising appropriate analogs, including without limitation, a PNA oligomer probe or an LNA oligomer probe, with or without a groove binder.

The term “extending enzyme” refers to a polypeptide that is able to catalyze the 5′-3′ extension of a hybridized primer in template-dependent manner under suitable reaction conditions including without limitation, appropriate nucleotide triphosphates, cofactors, buffer, and the like. Extending enzymes are typically DNA polymerases, for example but not limited to, RNA-dependent DNA polymerases, including without limitation reverse transcriptases, DNA-dependent DNA polymerases, and include DNA polymerases that, at least under certain conditions, share properties of both of these classes of DNA polymerases, including enzymatically active mutants or variants of each of these. In certain embodiments, an extending enzyme is a reverse transcriptase, including enzymatically active mutants or variants thereof, for example but not limited to, retroviral reverse transcriptases such as Avian Myeloblastosis Virus (AMV) reverse transcriptase and Moloney Murine Leukemia Virus (MMLV) reverse transcriptase. In certain embodiments, an extending enzyme is a DNA polymerase, including enzymatically active mutants or variants thereof. Certain DNA polymerases possess reverse transcriptase activity under some conditions, for example but not limited to, the DNA polymerase of Thermus thermophilus (Tth DNA polymerase, E.C. 2.7.7.7) which demonstrates reverse transcription in the presence of Mn²⁺, but not Mg²⁺ (see also, GeneAmp™ AccuRT RNA PCR Kit and Hot Start RNA PCR Kit comprising a recombinant polymerase derived from Thermus specie Z05, both from Applied Biosystems). Likewise, certain reverse transcriptases possess DNA polymerase activity under certain reaction conditions, including without limitation, AMV reverse transcriptase and MMLV reverse transcriptase. Descriptions of appropriate DNA polymerases for use with the disclosed methods and kits can be found in, among other places, Lehninger Principles of Biochemistry, 3d ed., Nelson and Cox, Worth Publishing, New York, N.Y., 2000 (“Lehninger”), particularly Chapters 26 and 29; R. M. Twyman, Advanced Molecular Biology: A Concise Reference. Bios Scientific Publishers, New York, N.Y. (1999); and Enzymatic Resource Guide: Polymerases, Promega, Madison, Wis. (1998). Expressly within the intended scope of the term extending enzyme are enzymatically active mutants or variants thereof, as are enzymes modified to confer different temperature-sensitive properties (see, e.g., U.S. Pat. Nos. 5,773,258; 5,677,152; and 6,183,998).

In certain embodiments, a primer, an Amplicon, or a primer and an Amplicon comprise a reporter group. In certain embodiments, a primer comprising a reporter group is incorporated into an Amplicon by primer extension. In certain embodiments, an Amplicon comprises a reporter group that was incorporated into the Amplicon when a reporter group-labeled dNTP was incorporated during primer extension or other amplification technique. A reporter group can, under appropriate conditions, emit a fluorescent, a chemiluminescent, a bioluminescent, a phosphorescent, or an electrochemiluminescent signal. Exemplary reporter groups include, but are not limited to fluorophores, radioisotopes, chromogens, enzymes, antigens including but not limited to epitope tags, semiconductor nanocrystals such as quantum dots, heavy metals, dyes, phosphorescence groups, chemiluminescent groups, electrochemical detection moieties, affinity tags, binding proteins, phosphors, rare earth chelates, transition metal chelates, near-infrared dyes, including but not limited to, “Cy7SPh.NCS,” “Cy7OphEt.NCS,” “Cy7OphEt.CO₂Su”, and IRD800 (see, e.g., J. Flanagan et al., Bioconjug. Chem. 8:751-56 (1997); and DNA Synthesis with IRD800 Phosphoramidite, LI-COR Bulletin #111, LI-COR, Inc., Lincoln, Nebr.), electrochemiluminescence labels, including but not limited to, tris(bipyridal) ruthenium (II), also known as Ru(bpy)₃²⁺, Os(1,10-phenanthroline) ₂bis(diphenylphosphino)ethane²⁺, also known as Os(phen)₂ (dppene)²⁺, luminol/hydrogen peroxide, Al(hydroxyquinoline-5-sulfonic acid), 9,10-diphenylanthracene-2-sulfonate, and tris(4-vinyl-4′-methyl-2,2′-bipyridal) ruthenium (II), also known as Ru(v-bpy₃²⁺), and the like.

The term reporter group also encompasses an element of multi-element indirect reporter systems, including without limitation, affinity tags such as biotin:avidin, antibody:antigen, ligand:receptor including but not limited to binding proteins and their ligands, and the like, in which one element interacts with one or more other elements of the system in order to effect the potential for a detectable signal. Exemplary multi-element reporter systems include an oligonucleotide comprising a biotin reporter group and a streptavidin-conjugated fluorophore, or vice versa; an oligonucleotide comprising a DNP reporter group and a fluorophore-labeled anti-DNP antibody; and the like. In certain embodiments, reporter groups, particularly multi-element reporter groups, are not necessarily used for detection, but serve as affinity tags for isolation/separation, for example but not limited to, a biotin reporter group and a streptavidin-coated Substrate, or vice versa; a digoxygenin reporter group and a substrate comprising an anti-digoxygenin antibody or a digoxygenin-binding aptamer; a DNP reporter group and a Substrate comprising an anti-DNP antibody or a DNP-binding aptamer; and the like. Detailed protocols for attaching reporter groups to oligonucleotides, oligonucleotides, peptides, antibodies and other proteins, mono-, di- and oligosaccharides, organic molecules, and the like can be found in, among other places, G. T. Hermanson, Bioconjugate Techniques, Academic Press, San Diego, 1996; Beaucage et al.; Molecular Probes Handbook; and Pierce Applications Handbook and Catalog 2003-2004, Pierce Biotechnology, Rockford, Ill., 2003 (“Pierce Applications Handbook”).

Multi-element interacting reporter groups are also within the scope of the term reporter group, such as fluorophore-quencher pairs, including without limitation fluorescent quenchers and dark quenchers (also known as non-fluorescent quenchers). A fluorescent quencher can absorb the fluorescent signal emitted from a fluorophore and after absorbing enough fluorescent energy, the fluorescent quencher can emit fluorescence at a characteristic wavelength, e.g., fluorescent resonance energy transfer. For example without limitation, the FAM-TAMRA pair can be illuminated at 492 nm, the excitation peak for FAM, and emit fluorescence at 580 nm, the emission peak for TAMRA. A dark quencher, appropriately paired with a fluorescent reporter group, absorbs the fluorescent energy from the fluorophore, but does not itself fluoresce. Rather, the dark quencher dissipates the absorbed energy, typically as heat. Exemplary dark or nonfluorescent quenchers include Dabcyl, Black Hole Quenchers, Iowa Black, QSY-7, AbsoluteQuencher, Eclipse non-fluorescent quencher, metal clusters such as gold nanoparticles, and the like. Certain dual-labeled probes comprising fluorophore-quencher pairs can emit fluorescence when the members of the pair are physically separated, for example but without limitation, nuclease probes such as TaqMan™ probes. Other dual-labeled probes comprising fluorophore-quencher pairs can emit fluorescence when the members of the pair are spatially separated, for example but not limited to hybridization probes, such as molecular beacons, or extension probes, such as Scorpion primers. Fluorophore-quencher pairs are well known in the art and used extensively for a variety of reporter probes (see, e.g., Yeung et al., BioTechniques 36:266-75, 2004; Dubertret et al., Nat. Biotech. 19:365-70, 2001; and Tyagi et al., Nat. Biotech. 18:1191-96, 2000).

In certain embodiments, a reporter group comprises an electrochemiluminescent moiety that can, under appropriate conditions, emit detectable electrogenerated chemiluminescence (ECL). In ECL, excitation of the electrochemiluminescent moiety is electrochemically driven and the chemiluminescent emission can be optically detected. Exemplary electrochemiluminescent reporter group species include: Ru(bpy)₃²⁺ and Ru(v-bpy)³²⁺ with emission wavelengths of 620 nm; Os(phen)₂ (dppene)²⁺ with an emission wavelength of 584 nm; luminol/hydrogen peroxide with an emission wavelength of 425 nm; Al(hydroxyquinoline-5-sulfonic acid) with an emission wavelength of 499 nm; and 9,10-diphenylanothracene-2-sulfonate with an emission wavelength of 428 nm; and the like. Forms of these three electrochemiluminescent reporter group species that are modified to be amenable to incorporation into probes are commercially available or can be synthesized without undue experimentation using techniques known in the art. For example, a Ru(bpy)₃²⁺ N-hydroxy succinimide ester for coupling to nucleic acid sequences through an amino linker group has been described (see, U.S. Pat. No. 6,048,687); and succinimide esters of Os(phen)₂ (dppene)²⁺ and Al(HQS)₃³⁺ can be synthesized and attached to nucleic acid sequences using similar methods. The Ru(bpy)₃²⁺ electrochemiluminescent reporter group can be synthetically incorporated into nucleic acid sequences using commercially available ru-phosphoramidite (IGEN International, Inc., Gaithersburg, Md.) (see, e.g., Osiowy, J. Clin. Micro. 40:2566-71, 2002).

Additionally other polyaromatic compounds and chelates of ruthenium, osmium, platinum, palladium, and other transition metals have shown electrochemiluminescent properties. Detailed descriptions of ECL and electrochemiluminescent moieties can be found in, among other places, A. Bard and L. Faulkner, Electrochemical Methods, John Wiley & Sons (2001); M. Collinson and M. Wightman, Anal. Chem. 65:2576 (1993); D. Brunce and M. Richter, Anal. Chem. 74:3157 (2002); A. Knight, Trends in Anal. Chem. 18:47 (1999); B. Muegge et al., Anal. Chem. 75:1102 (2003); H. Abrunda et al., J. Amer. Chem. Soc. 104:2641 (1982); K. Maness et al., J. Amer. Chem. Soc. 118:10609 (1996); M. Collinson and R. Wightman, Science 268:1883 et seq. (1995); and U.S. Pat. No. 6,479,233 (see also, O'Sullivan et al., Nucl. Acids Res. 30:e114, 2002 for a discussion of phosphorescent lanthanide and transition metal reporter groups).

### II. TECHNIQUES

The methods of the invention are directed to quantitating known oligonucleotides of interest, particularly but not limited to, small RNA molecules such as miRNA, siRNA, stRNA, and other ncRNA. In such methods, the sequence of the target oligonucleotide is known and first primer sets (e.g., reverse transcriptase, forward, reverse primers) and reporter probes can be designed based on the known sequence. Second primer sets can be designed to serve as: (i) amplification primers for individual first amplicons and additional first amplicons and may or may not encode target-specific hybridization tags, useful for subsequent isolation and/or identification, (ii) universal primers, for example but not limited to, multiplexed amplification of a multiplicity of first amplicons and/or additional first amplicons, typically in a uniform manner, or (iii) a combination of a universal primer and a target-specific primer that encodes a target-specific hybridization tag.

Other disclosed methods are directed to identifying unknown target oligonucleotides, particularly but not limited to, small RNA molecules such as miRNA, siRNA, stRNA, and other ncRNA. The sequence of interest is not known, although partially sequence information may be known or predicted. For illustration purposes but not as a limitation, several miRNA predictive algorithms are available (see, e.g., MiRscan, available on the web at genes/mitedu/mirscan; miRseeker; and Carter et al., Nucl. Acids Res. 29(19):3928-38, 2001). The scientific literature and available databases (see, e.g., the miRNA Registry, on the world-wide web at sanger-ac.uk/Software/Rfam/miRNA/index) can be analyzed to identify possible regions of homology, at one or both ends of potential miRNA targets that can be further evaluated using routine experimentation. Bioinformatics searching of the gDNA for possible stem-loop structures can also indicate potential miRNA targets for evaluation according to the current teachings. Additionally, unknown sequences can be identified empirically using the disclosed methods and compositions. In some embodiments, one or both primers of a first primer set for identifying a oligonucleotide target, including without limitation, a small RNA molecule, comprise a oligonucleotide-binding portion including at least 2, 3, 4, 5, 6, or 7 random or degenerate nucleotides, including without limitation, a universal base. The invention is based in part on the discovery that the design of the reverse transcriptase primer is essential to the sensitivity of the method. The design of the reverse transcriptase (RT) primer plays an important role in obtaining a large dynamic range. The RT-primer can be divided into three sections, namely:
A. SRS-sequence (SiRNA Related Sequence)
B. Probe sequence
C. Reverse primer sequence

### A. The SRS-Sequence:

Reverse Transcriptase is able to transcribe a RNA or DNA molecule into cDNA when as few as two nucleotides are present that are complementary to the last two nucleotides at the 3′-end of the RT-primer. Particular rules apply to the design of the reverse transcriptase primer. It is important to (1) Prevent in the RT-primer design that the 3′-end of the SRS-sequence ends with the sequence combination GC, CG, AT or TA as the RT-primer is able to form a dimer and transcribe itself. To avoid this, if the 3′-end has any of the above sequences, one should extend or shorten the SRS-sequence so it ends with GT, GA, CT or CA; (2) Prevent in the RT-primer design complementary repetition(s) to the 3′-end of the SRS-sequence. For example: if the 3′-end encodes GT, do not allow AC to occur anywhere within the RT-primer. If the AC-sequence is located within the SRS-sequence, extend or shorten the SRS-sequence; and (3) The SRS-sequence can vary in length ranging from as few as 2 to 11 nucleotides. For example, it is possible that the SRS sequence covers most of the siRNA sequence, e.g., if the siRNA is 19 nucleotides long, the SRS sequence can be 17 nucleotides long.

### B. Probe Sequence.

The probe sequence can vary from 17 to 30 nucleotides in size. The sequence can be complementary to the sense or anti-sense strand. The probe sequence is not restricted to the RT-primer but can also span part of the siRNA sequence. The probe can be labeled with different fluorescent labels such as VIC, JOE, TAMRA, FAM, CY3, CY5, and the like, in combination with different quenchers such as TAMRA, BHQ and the like. The use of a specific labeled probe within the PCR allows for multiplex RT-PCR. This allows for simultaneous measurements of, for example the siRNA levels, siRNA-target mRNA levels and an internal control, in the same biological sample.

### C. Reverse Primer Sequence.

The Reverse primer sequence can vary in length but has to produce a unique PCR product when used in combination with the forward primer.

When designing the primers for the reaction, there no sequence overlap between the 3′-terminus of the reverse primer and the 3′-terminus of the reverse transcription primer. With respect to the forward primer and the reverse transcription primer, if there is too much sequence overlap, this will result in amplification of the revere transcription primer in a template independent manner. As few as 3 nucleotides overlap between the 3′-terminus of the forward primer and the reverse primer is sufficient. In order to prevent this, one should minimize the overlap to a maximum of 2 nucleotides overlap.

While the certain embodiments of these methods employ “RT-PCR-PCR like” amplification techniques, other amplification techniques are also contemplated. Further, certain embodiments of the disclosed methods comprise a single reaction composition in which Amplicons are generated. Other embodiments comprise two or more reaction compositions, including without limitation, a multiplex format comprising a first reaction composition in which first products, first amplicons and additional first amplicons are generated, and a multiplicity of different second reaction compositions in which second amplicons are generated.

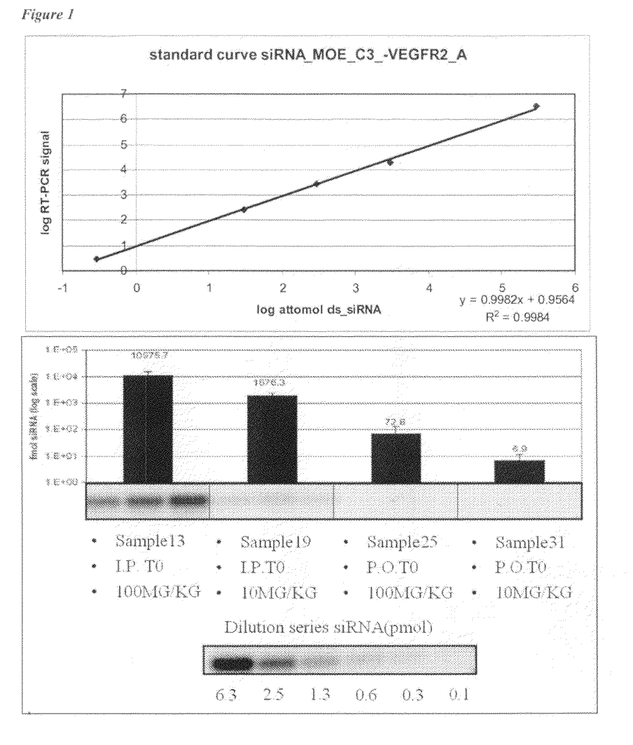

An overview of some aspects of certain disclosed methods is depicted in FIG. 1 for illustration purposes, but is not intended to limit the current teachings in any way. An exemplary siRNA target hybridizes to a corresponding reverse transcription primer of a first primer set and in the presence of an extending enzyme, the hybridized reverse transcription primer is extended and a single strand copy DNA is formed. Those in the art will appreciate that according to conventional methodology, the double-stranded siRNA is denatured before the reverse transcription primer can bind (for example, but not limited to, 5 minutes at 95° C.), often in a thermocycler. Surprisingly, the inventors have observed that when the target is a double-stranded siRNA, the reverse transcription primer can be incorporated isothermally, i.e., without a denaturation step. Without being limited to a particular theoretical basis, this may be due to the concentration of the siRNA-first target duplex (typically in the 10⁻⁵ (fM) to 10⁻¹² (pM) range) relative to the concentration of the first primer set (typically in the 10⁻⁸ (nM) to 10⁻⁶ (microM) range). Under these conditions, the reverse transcription primer might displace 5′-end of the siRNA-duplex and be extended by an extending enzyme, even at sub-optimum temperatures for enzyme activity. For example, in certain embodiments wherein the target is a small RNA molecule, the first reaction composition is incubated at about 20° C. for several minutes (for example, but not limited to 10-30 minutes) and then the temperature is raised to optimize or at least enhance the activity of the extending enzyme (typically a reverse transcriptase in such an embodiment). Thus, in certain embodiments, a denaturation step is included prior to the step of generating single strand copy DNA, while in other embodiments, it is optional. The temperature of the reaction composition is raised to inactivate the reverse transcriptase (if any) and/or to activate a second extending enzyme, if appropriate (for example, a “hot start” polymerase).

In a second reaction, under appropriate reaction conditions, the forward primer hybridizes with the single strand copy DNA, is extended by a second extending enzyme (for example, a “hot start” polymerase) and a fust amplicon is formed. In a second step, after the reaction composition is denatured (for example, but not limited to, 95° C. or above for 10-20 second), the temperature is lowered (for example, but not limited to, about 60° C. for approximately 1 minute) allowing the reverse primer to hybridizes with the first amplicon and the forward primer to the single strand copy DNA, followed by extension of both primers by the second extending enzyme. The reaction composition is then cycled between denaturation temperatures and annealing/extension temperatures (for example, but not limited to, 95° C. or above for 10-20 second, then about 60° C. for approximately 1, minute) for a limited number of cycles (for example, but not limited to, 35 to 50 cycles) to generate first amplicons and additional second amplicons.

In certain embodiments, after the first amplicons and the additional first amplicons are generated, a second primer set and optionally, an extending enzyme are added to form a second reaction composition. In other embodiments, discussed below, the second primer set(s) are included in the first reaction composition. The reaction composition is heated to a temperature sufficient to denature the first amplicons and the additional first amplicons. The reaction composition is cooled to allow the primers of the second primer set to hybridize to the separated strands of the first amplicons or the additional first amplicons and the hybridize primers of the second primer set are extending by the extending enzyme to generate second amplicons and the cycle is repeated as necessary.

In one embodiment, the forward and reverse primers are unmodified primers. In another embodiment, the forward or reverse primers can be modified. Such modifications help to increase affinity and/or specificity of the primers for the target. Examples of modifications include, but are not limited to, 2′ alkoxyribonucleotide, 2′ alkoxyalkoxy ribonucleotide, a locked nucleic acid ribonucleotide (LNA), 2′-fluoro ribonucleotide, morpholino nucleotide.

In another embodiment, the modified nucleotide is selected from among nucleotides having a modified internucleoside linkage selected from among phosphorothioate, phosphorodithioate, phosphoramidate, boranophosphonoate, and amide linkages.

In certain embodiments, a reporter probe is added to the second reaction composition when the second primer set and optional extending enzyme are added. In other embodiments, reporter probes are added at a later step. Those in the art will appreciate that when detection comprises using reporter probes in a nuclease assay including but not limited to a TaqMan™ assay, or a probe extension assay, an appropriate DNA polymerase (which may or may not be the same as the second extending enzyme) needs to be included in the reaction composition. The reaction is cycled, depending on the reporter probes and the nature of the detection assay employed, and the reporter probes (for example but not limited to cleaved reporter groups) are detected and the corresponding target is identified or quantitated.

Those in the art will appreciate that detection can comprise a variety of reporter probes with different mechanisms of action and that detection can be performed either in real-time or at an end-point. It will also be appreciated that detection can comprise reporter groups that are incorporated into the Amplicons, either as part of labeled primers or due to the incorporation of labeled dNTPs during an amplification, or attached to Amplicons, for example but not limited to, via hybridization tag complements comprising reporter groups or via linker arms that are integral or attached to Amplicons.

In certain embodiments of the disclosed methods, a single reaction composition is formed and two, three or four amplification steps (depending on the reaction format) occur in the same reaction composition and typically, the same reaction vessel. According to certain embodiments of the disclosed methods, a first reaction composition comprises a oligonucleotide target, a first primer set, and an extending enzyme; and a first product, a first amplicon, an additional first amplicon, or combinations thereof, are generated and detected; and the target oligonucleotide is identified and/or quantitated.

In certain embodiments, the single reaction composition further comprises a second primer set. The first and second primers of the second primer set are used to amplify the first amplicon and/or additional first amplicon to generate a second amplicon. In certain embodiments, a primer of the second primer set is a universal primer. In certain embodiments, both primers of at the second primer set comprise universal primers. In certain embodiments, one of the second primers is a universal primer and the corresponding primer comprises a hybridization tag that typically encodes a target-specific sequence that can be subsequently used to correlate the second amplicon to its corresponding oligonucleotide target. In certain embodiments, a primer of the second primer set comprises an affinity tag. In certain embodiments, the second amplicon is cycled with additional primers of the second primer set to generate more second amplicons. In certain embodiments, the second amplicons or their surrogates are detected and the corresponding oligonucleotide target is identified and/or quantitated.

In certain embodiments, a oligonucleotide target comprises a small RNA molecule, the extending enzyme comprises a reverse transcriptase or a DNA polymerase with reverse transcriptase activity, and the first product comprises a reverse-transcribed product. In certain embodiments, at least two different extending enzymes are used, including a reverse transcriptase and a DNA polymerase.

In certain embodiments, the disclosed methods comprise forming at least two different reaction compositions. In essence, two primer sets per oligonucleotide target are used in three or four amplification steps that occur in two different reaction compositions and can, but need not, take place in the same reaction vessel. The amplification steps that typically occur include: hybridizing a reverse transcription primer to the oligonucleotide molecule, wherein the reverse transcription primer comprises an oligonucleotide molecule-binding portion having an oligonucleotide recognition sequence comprising at least 2 nucleotides at the 3′ region that are complementary to a region of the oligonucleotide molecule and an extension tail comprising at least 2 nucleotides at the 5′ region; extending the hybridized reverse transcription primer with a first extending enzyme to generate a reverse-transcribed product; hybridizing a forward primer to the reverse-transcribed product, wherein the forward primer comprises an oligonucleotide molecule-binding portion comprising at least 2 nucleotides that are the same as a region of the oligonucleotide molecule; extending the hybridized forward primer with a second extending enzyme to generate a first amplicon; hybridizing a reverse primer to the first amplicon; extending the hybridized reverse primer with the second extending enzyme to generate a second amplicon complementary to the first amplicon; detecting the amplification product; and thereby identifying or quantifying the oligonucleotide molecule. The reaction can, but need not, comprise real-time detection. In certain embodiments, an amplification step comprises multiplexing.

A oligonucleotide target according to the present teachings may be derived from any living, or once living, organism, including but not limited to, prokaryotes, archaca, viruses, and eukaryotes. The oligonucleotide target can also be synthetic. The oligonucleotide target may originate from the nucleus, typically genomic DNA (gDNA) and RNA transcription products (including without limitation certain miRNA precursors and other small RNA molecules), or may be extranuclear, e.g., cytoplasmic, plasmid, mitochondrial, viral, etc. The skilled artisan appreciates that gDNA includes not only full length material, but also fragments generated by any number of means, for example but not limited to, enzyme digestion, sonication, shear force, and the like. In certain embodiments, the oligonucleotide target may be present in a double-stranded or single-stranded form.

A variety of methods are available for obtaining a oligonucleotide target for use with the methods and kits of the present teachings. When the target sequences are obtained from a biological matrix, certain isolation techniques are typically employed, including without limitation, (1) organic extraction followed by ethanol precipitation, e.g., using a phenol/chloroform organic reagent (see, e.g., Ausbel et al., particularly Volume 1, Chapter 2, Section I), in certain embodiments, using an automated extractor, e.g., the Model 341 DNA Extractor (Applied Biosystems); (2) stationary phase adsorption methods (see, e.g., U.S. Pat. No. 5,234,809; Walsh et al., BioTechniques 10(4): 506-513 (1991)); and (3) salt-induced DNA precipitation methods (see, e.g., Miller et al., Nucl. Acids Res. 16(3): 9-10, 1988), such precipitation methods being typically referred to as “salting-out” methods. In certain embodiments, the above isolation methods may be preceded by an enzyme digestion step to help eliminate unwanted protein from the sample, e.g., digestion with proteinase K, or other like proteases. See, e.g., U.S. patent application Ser. No. 09/724,613; see also, U.S. patent application Ser. Nos. 10/618,493 and 10/780,963; and U.S. Provisional Patent Application Ser. Nos. 60/499,082 and 60/523,056. A variety of commercially available kits and instruments can also be used to obtain target oligonucleotides, including but not limited to small RNA molecules and their precursors, for example but not limited to, the ABI PRISM™. TransPrep System, BloodPrep™. Chemistry, ABI PRISM™ 6100 Nucleic Acid PrepStation, and ABI PRISM™ 6700 Automated Nucleic Acid Workstation (all from Applied Biosystems); the SV96 Total RNA Isolation System and RNAgents™. Total RNA Isolation System (Promega, Madison, Wis.); the mirVana miRNA Isolation Kit (Ambion, Austin, Tex.); and the Absolutely RNA™ Purification Kit and the Micro RNA Isolation Kit (Stratagene, La Jolla, Calif.).

In certain embodiments, oligonucleotide molecules in a sample may be subjected to restriction enzyme cleavage and the resulting restriction fragments may be employed as oligonucleotide targets. Different oligonucleotide targets may be different portions of a single contiguous nucleic acid or may be on different nucleic acids. Different target sequences of a single contiguous nucleic acid may or may not overlap. Certain oligonucleotide targets may also be present within other target sequences, including without limitation, primary miRNA (pri-miRNA), precursor miRNA (pre-miRNA), miRNA, mRNA, and siRNA.

Certain embodiments of the disclosed methods comprise a step for generating a first product, a step for generating a first amplicon, a step for generating additional first amplicons, a step for generating second amplicons, a step for generating more second amplicons, or combinations thereof. In certain embodiments, at least some of these steps occur simultaneously or nearly simultaneously in a first reaction composition. In certain embodiments, some of these steps occur in a first reaction composition and other steps occur in a second reaction composition or a third reaction composition. Certain kits of the current teachings comprise an amplification means.

Amplification according to the present teachings encompasses any means by which at least a part of a target oligonucleotide and/or an Amplicon is reproduced, typically in a template-dependent manner, including without limitation, a broad range of techniques for amplifying nucleic acid sequences, either linearly or exponentially. Exemplary techniques for performing an amplifying step include the polymerase chain reaction (PCR), primer extension (including but not limited to reverse transcription), strand displacement amplification (SDA), multiple displacement amplification (MDA), nucleic acid strand-based amplification (NASBA), rolling circle amplification (RCA), transcription-mediated amplification (TMA), transcription, and the like, including multiplex versions or combinations thereof. Descriptions of such techniques can be found in, among other places, Sambrook and Russell; Sambrook et al.; Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); Rapley; U.S. Pat. Nos. 6,027,998 and 6,511,810; PCT Publication Nos. WO 97/31256 and WO 01/92579; Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000).

In certain embodiments, amplification comprises a cycle of the sequential steps of: (i) hybridizing a primer with a target oligonucleotide and/or an Amplicon comprising complementary or substantially complementary sequences; (ii) extending the hybridized primer, thereby synthesizing a strand of nucleotides in a template-dependent manner; and (iii) denaturing the newly-formed nucleic acid duplex to separate the strands. The cycle may or may not be repeated, as desired. Amplification can comprise thermocycling or can be performed isothermally. In certain embodiments, nascent nucleic acid duplexes are not initially denatured, but are used in their double-stranded form in one or more subsequent steps and either one or both strands can, but need be, detected. In certain embodiments, single-stranded Amplicons are generated, for example but not limited to, asymmetric PCR.

Primer extension is an amplifying technique that comprises elongating a primer that is annealed to a template in the 5′=>3′ direction using an amplifying means such as an extending enzyme, for example but not limited to, a DNA polymerase (including without limitation, a reverse transcriptase). According to certain embodiments, with appropriate buffers, salts, pH, temperature, and nucleotide triphosphates, including analogs thereof, an extending enzyme incorporates nucleotides complementary to the template strand starting at the 3′-end of an annealed primer, to generate a complementary strand. In certain embodiments, the extending enzyme used for primer extension lacks or substantially lacks 5′-exonuclease activity.

The skilled artisan will understand that a number of different enzymes, including without limitation, extending enzymes could be used in the disclosed methods and kits, for example but not limited to, those isolated from thermostable or hyperthermostable prokaryotic, eukaryotic, or archaeal organisms. The skilled artisan will also understand that enzymes such as polymerases, including but not limited to DNA-dependent DNA polymerases and RNA-dependent DNA polymerases, include not only naturally occurring enzymes, but also recombinant enzymes; and enzymatically active fragments, cleavage products, mutants, or variants of such enzymes, for example but not limited to Klenow fragment, Stoffel fragment, Taq FS (Applied Biosystems), 9 Nₘ™. DNA Polymerase (New England BioLabs, Beverly, Mass.), and mutant enzymes (including without limitation, naturally-occurring and man-made mutants), described in Luo and Barany, Nucl. Acids Res. 24:3079-3085 (1996), E is et al., Nature Biotechnol. 19:673-76 (2001), and U.S. Pat. Nos. 6,265,193 and 6,576,453. Reversibly modified polymerases, for example but not limited to those described in U.S. Pat. No. 5,773,258, are also within the scope of the disclosed teachings. The present teachings also contemplate various uracil-based decontamination strategies, wherein for example uracil can be incorporated into an amplification reaction, and subsequent carry-over products removed with various glycosylase treatments (see, e.g., U.S. Pat. No. 5,536,649). Those in the art will understand that any protein with the desired enzymatic activity can be used in the disclosed methods and kits. Descriptions of DNA polymerases, including reverse transcriptases, uracil N-glycosylase, and the like, can be found in, among other places, Twyman, Advanced Molecular Biology, BIOS Scientific Publishers, 1999; Enzyme Resource Guide, rev. 092298, Promega, 1998; Sambrook and Russell; Sambrook et al.; Lehninger; PCR: The Basics; and Ausbel et al.

Certain embodiments of the disclosed methods and kits comprise separating (either as a separate step or as part of a step for detecting) or a separation means. Separating comprises any process that removes at least some unreacted components or at least some reagents from an Amplicon. In certain embodiments, Amplicons are separated from unreacted components and reagents, including without limitation, unreacted molecular species present in a reaction composition, extending enzymes, primers, co-factors, dNTPs, and the like. The skilled artisan will appreciate that a number of well-known separation means can be used in the methods and kits disclosed herein and thus the separation technique employed is not a limitation on the disclosed methods.

Exemplary means/techniques for performing a separation step include gel electrophoresis, for example but not limited to, isoelectric focusing and capillary electrophoresis; dielectrophoresis; flow cytometry, including but not limited to fluorescence-activated sorting techniques using beads, microspheres, or the like; liquid chromatography, including without limitation, HPLC, FPLC, size exclusion (gel filtration) chromatography, affinity chromatography, ion exchange chromatography, hydrophobic interaction chromatography, immunoaffinity chromatography, and reverse phase chromatography; affinity tag binding, such as biotin-avidin, biotin-streptavidin, maltose-maltose binding protein (MBP), and calcium-calcium binding peptide; aptamer-target binding; hybridization tag-hybridization tag complement annealing; mass spectrometry, including without limitation MALDI-TOF, MALDI-TOF-TOF, tandem mass spec (MS-MS), LC-MS, and LC-MS/MS; a microfluidic device; and the like: Discussion of separation techniques and separation-detection techniques, can be found in, among other places, Rapley; Sambrook et al.; Sambrook and Russell; Ausbel et al.; Molecular Probes Handbook; Pierce Applications Handbook; Capillary Electrophoresis: Theory and Practice, P. Grossman and J. Colburn, eds., Academic Press, 1992; The Expanding Role of Mass Spectrometry in Biotechnology, G. Siuzdak, MCC Press, 2003; PCT Publication No. WO 01/92579; and M. Ladisch, Bioseparations Engineering: Principles, Practice, and Economics, John Wiley & Sons, 2001.

In certain embodiments, detecting step comprises separating and/or detecting an Amplicon using an instrument, i.e., using an automated or semi-automated detection means that can, but need not, comprise a computer algorithm. In certain embodiments, the detection step is combined with or is a continuation of a separating step, for example but not limited to a capillary electrophoresis instrument comprising a fluorescent scanner and a graphing, recording, or readout component; a capillary electrophoresis instrument coupled with a mass spectrometer; a chromatography column coupled with an absorbance monitor or fluorescence scanner and a graph recorder, or with a mass spectrometer; or a microarray with a data recording device such as a scanner or CCD camera. In certain embodiments, the detecting step is combined with the amplifying step and the quantifying and/or identifying step, for example but not limited to, real-time analysis such as Q-PCR. Exemplary means for performing a detecting step include capillary electrophoresis instruments, for example but not limited to, the ABI PRISM™. 3100 Genetic Analyzer, ABI PRISM™ 3100-Avant Genetic Analyzer, ABI PRISM™ 3700 DNA Analyzer, ABI PRISM™ 3730 DNA Analyzer, ABI PRISM™ 3730×/DNA Analyzer (all from Applied Biosystems); the ABI PRISM™ 7300 Real-Time PCR System; the ABI PRISM™ 7700 Sequence Detection System; mass spectrometers; and microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:140-45, including supplements, 2003). Exemplary software for reporter group detection, data collection, and analysis includes GeneMapper™ Software, GeneScan™ Analysis Software, and Genotyper™ software (all from Applied Biosystems).

In certain embodiments, separating or detecting comprises flow cytometry methods, including without limitation fluorescence-activated sorting (see, e.g., Vignali, J. Immunol. Methods 243:243-55, 2000). In certain embodiments, detecting comprises: separating an Amplicon and/or an Amplicon surrogate using a mobility-dependent analytical technique, such as capillary electrophoresis; monitoring the eluate using, for example but without limitation, a fluorescent scanner, to detect the Amplicons as they elute; and evaluating the fluorescent profile of the Amplicons, typically using detection and analysis software, such as an ABI PRISM™ Genetic Analyzer using GeneScan™ Analysis Software (both from Applied Biosystems). In certain embodiments, determining comprises a plate reader and an appropriate illumination source.

In certain embodiments, detecting comprises a single-stranded Amplicon or Amplicon surrogate, for example but not limited to, detecting a reporter group that is integral to the single-stranded molecule being detected, such as a fluorescent reporter group that is incorporated into an Amplicon or the reporter group of a released hybridization tag complement (an exemplary Amplicon surrogate); a reporter group on a molecule that hybridizes with the single-stranded Amplicon being detected, such as a reporter probe.

In certain embodiments, a double-stranded Amplicon is detected. Typically such double-stranded Amplicons or Amplicon surrogates are detected by triplex formation or by local opening of the double-stranded molecule, using for example but without limitation, a PNA opener, a PNA clamp, and triplex forming oligonucleotides (TFOs), either reporter group-labeled or used in conjunction with a labeled entity such as a molecular beacon (see, e.g., Drewe et al., Mol. Cell. Probes 14:269-83, 2000; Zelphati et al., BioTechniques 28:304-15, 2000; Kuhn et al., J. Amer. Chem. Soc. 124:1097-1103, 2002; Knauert and Glazer, Hum. Mol. Genet. 10:2243-2251, 2001; Lohse et al., Bioconj. Chem. 8:503-09, 1997). In certain embodiments, an Amplicon and/or an Amplicon surrogate comprises a stretch of homopurine sequences.

### III MODIFIED NUCLEOTIDES

In one embodiment, the invention features a chemically-modified nucleic acid molecules, e.g., short interfering nucleic acid molecules, wherein the chemical modification comprises a conjugate covalently attached to the nucleic acid molecule. Non-limiting examples of conjugates include, but are not limited to 2′ alkoxyribonucleotide, 2′ alkoxyalkoxy ribonucleotide, a locked nucleic acid ribonucleotide (LNA), 2′-fluoro ribonucleotide, morpholino nucleotide. In another embodiment, the modified nucleotide is selected from among nucleotides having a modified internucleoside linkage selected from among phosphorothioate, phosphorodithioate, phosphoramidate, boranophosphonoate, and amide linkages.

In one embodiment, a conjugate molecule of the invention comprises a molecule that facilitates delivery of the chemically-modified nucleic acid molecule, e.g., siRNA molecule into a biological system, such as a cell. In another embodiment, the conjugate molecule attached to the chemically-modified siRNA molecule is a polyethylene glycol, human serum albumin, or a ligand for a cellular receptor that can mediate cellular uptake. Examples of specific conjugate molecules are described in Vargeese et al., U.S. Ser. No. 10/201,394, filed Jul. 22, 2002 incorporated by reference herein. The type of conjugates used and the extent of conjugation of siRNA molecules of the invention can be evaluated for improved pharmacokinetic profiles, bioavailability, and/or stability of the siRNA constructs while at the same time maintaining the ability of the siRNA to mediate RNAi activity. As such, one skilled in the art can screen siRNA molecules that are modified with various conjugates to determine whether the siRNA conjugate complex possesses improved properties while maintaining the ability to mediate RNAi, for example in animal models as are generally known in the art. The chemically-modified nucleic acid molecule can also be formulated with a pharmaceutical carrier capable of facilitating delivery to and/or uptake by the target cells. Selected from, but not limited to, neutral liposomes, cationic liposomes or lipoplexes, cationic polymers or polyplexes, neutral polymers, nanoparticles, double stranded RNA binding proteins, calcium phosphate, cell penetrating peptides, viral proteins and viral particles, antibodies and empty bacterial envelopes.

In one embodiment, the invention features a short interfering nucleic acid siRNA molecule which comprises a nucleotide, non-nucleotide, or mixed nucleotide/non-nucleotide, e.g., an aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art. (See, for example, Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.)

Examples of modifications include, but are not limited to, modification of the cap region. By “cap structure” is meant chemical modifications, which have been incorporated at either terminus of the oligonucleotide (see, for example, Adamic et al., U.S. Pat. No. 5,998,203, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples, the 5′-cap includes, but is not limited to, glyceryl, inverted deoxy abasic residue (moiety); 4′,5′-methylene nucleotide; 1-(beta-D-erythrofuranosyl)nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl)nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediolphosphate; 5′-amino; bridging and/or non-bridging 5′-, phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

In another embodiment, the invention features conjugates and/or complexes of siRNA molecules of the invention. Such conjugates and/or complexes can be used to facilitate delivery of siRNA molecules into a biological system, such as a cell. The conjugates and complexes provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel conjugates and complexes for the delivery of molecules, including, but not limited to, small molecules, lipids, cholesterol, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

Therapeutic nucleic acid molecules (e.g., siRNA molecules) delivered exogenously optimally are stable within cells until reverse transcription of the RNA has been modulated long enough to reduce the levels of the RNA transcript. The nucleic acid molecules are resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In yet another embodiment, siRNA molecules having chemical modifications that maintain or enhance enzymatic activity of proteins involved in RNAi are provided. Such nucleic acids are also generally more resistant to nucleases than unmodified nucleic acids. Thus, in vitro and/or in vivo the activity should not be significantly lowered.

Use of the nucleic acid-based molecules of the invention will lead to better treatment of the disease progression by affording the possibility of combination therapies (e.g., multiple siRNA molecules targeted to different genes; nucleic acid molecules coupled with known small molecule modulators; or intermittent treatment with combinations of molecules, including different motifs and/or other chemical or biological molecules). The treatment of subjects with siRNA molecules can also include combinations of different types of nucleic acid molecules, such as enzymatic nucleic acid molecules (ribozymes), allozymes, antisense, 2,5-A oligoadenylate, decoys, and aptamers.

In another aspect a siRNA molecule of the invention comprises one or more 5′ and/or a 3′-cap structure, for example on only the sense siRNA strand, the antisense siRNA strand, or both siRNA strands.

### IV. DELIVERY OF NUCLEIC ACID MOLECULES

Nucleic acid molecules, e.g., siRNA molecules can be adapted for use to modulate, ameliorate, or treat, for example, variety of disease and conditions described herein, such as proliferative diseases and conditions and/or cancer including breast cancer, cancers of the head and neck including various lymphomas such as mantle cell lymphoma, non-Hodgkins lymphoma, adenoma, squamous cell carcinoma, laryngeal carcinoma, cancers of the retina, cancers of the esophagus, multiple myeloma, ovarian cancer, uterine cancer, melanoma, colorectal cancer, lung cancer, bladder cancer, prostate cancer, glioblastoma, lung cancer (including non-small cell lung carcinoma), pancreatic cancer, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, ocular diseases such as macular degeneration (e.g., wet/dry AMD), corneal neovascularization, diabetic retinopathy, neovascular glaucoma, myopic degeneration and other proliferative diseases and conditions such as restenosis and polycystic kidney disease, and any other diseases or conditions that are related to or will respond to the levels of the target protein, e.g., VEGF and/or VEGFr in a cell or tissue, alone or in combination with other therapies. For example, a siRNA molecule can comprise a delivery vehicle, including liposomes, for administration to a subject, carriers and diluents and their salts, and/or can be present in pharmaceutically acceptable formulations. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129 140; Hofland and Huang, 1999, Handb. Exp. Pharmacol., 137, 165 192; and Lee et al., 2000, ACS Symp. Ser., 752, 184 192, all of which are incorporated herein by reference. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis (see for example WO 03/043689 and WO 03/030989), or by incorporation into other vehicles, such as biodegradable polymers, hydrogels, cyclodextrins (see for example Gonzalez et al., 1999, Bioconjugate Chem., 10, 1068 1074; Wang et al., International PCT publication Nos. WO 03/47518 and WO 03/46185), poly(lactic-co-glycolic)acid (PLGA) and PLCA microspheres (see for example U.S. Pat. No. 6,447,796 and U.S. Patent Application Publication No. U.S. 2002130430), biodegradable nanocapsules, and bioadhesive microspheres, or by proteinaceous vectors (O'Hare and Normand, International PCT Publication No. WO 00/53722). In another embodiment, the nucleic acid molecules of the invention can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives. Alternatively, the nucleic acid/vehicle combination is locally delivered by direct injection or by use of an infusion pump.

Further methods to increase the efficiency of in vivo oligonucleotide administration into vertebrates involve using chemical agents or physical manipulations. Such chemical agents include polymers (Mumper, R. J., et al., Pharm. Res. 13:701-709 (1996); Mumper R. J., et al., J. Cont. Rel. 52:191-203 (1998); Anwer, K., et al., Pharm. Res, 16:889-895 (1999); Boussif O., et al., Proc. Natl. Acad. Sci. USA 92:7297-7301 (1995); Orson F. M., et al., J. Immunol. 164:6313-6321 (2000); Turunen M. P., et al., Gene Ther. 6:6-11 (1999); Shi N. Y., et al., Proc. Natl. Acad. Sci. USA. 97:7567-7572 (2000); Rozema, D. B. PNAS early edition July 24, 1-6 (2007); Thomas, M. et al Expet opin. Biol Ther. 5:495-505 (2005); Howard, K. A. et al Mol. Ther. 14:476-484 (2006); Leong K. W., et al., J. Controlled Release 53:183-193 (1998); Baranov A., et al., Gene Ther. 6:1406-1414 (1999); Lunsford L., et al., J. Drug Targeting 8:39-50 (2000); Bertling W. M., et al., Biotechnol. Appl. Biochem. 13:390-405 (1991); Heldel J. D., PNAS. 104:5715-5721 (2007); Schiffelers, R. M. et al Nucleic Acids Res. 32:e149 (2004); Davis, M. E. et al Curr. Med. Chem. 11:179-197 (2004)), detergents (Freeman D. J. and Niven R. W., Pharm. Res. 13:202-209 (1996); Raczka E., et al. Gene Ther. 5:1333-1339 (1998)), cationic or non-cationic lipids that may facilitate oligonucleotide entry into lipid bilayers of cells (Liu Y., et al., Nat. Biotechnol. 15:167-173 (1997); Eastman S. J., et al. Hum. Gene Ther. 8:313-322 (1997); Simoes, S., et al., Biochim. Biophys. Acta Biomembranes 1463:459-469 (2000); Thierry, A. R., et al., Gene Ther. 4:226-237 (1997); Floch V., et al. Biochim. Biophys. Acta Biomembranes 1464:95-103 (2000); Egilmez N. K., et al. Biochem. Biophys. Res. Commun. 221:169-173 (1996); Santel A., Gene Ther. 13:1360-1370 (2006); Li, W. Pharm. Res. 24:438-449 (2007), Pirollo, K. F. Cancer Res. 67:(7) 2938-2943 (2007); Cardoso, A. L. C. J. Gene Med. 9:170-183 (2007); Zimmermann, T. S. et al Nature 441:111-114 (2006); Chem, P. Y et al Cancer Gene Ther. 2:321-328); Landen, C. N. et al Cancer Res. 65:6910-6918 (2005); Morrissey, D. V. et al Nat. Biotechno. 23:1002-1007 (2005)), proteins and peptides (Ryter J. M., The EMBO Journal 17:7505-7515 (1998); Kumar, P. et al. Nature 448: 39-43 (2007); Deshayes, S. et al Biochimica Acta, 1667:141-147 (2004); Morris, M. C. et al Nucleic acids Research 25:2730-2736 (1997); Simeoni, F. et al Nucleic acids Research 31:2717-2724 (2003); U.S. Pat. Nos. 5,264,618 and 5,334,761. Additional methods involve using empty bacterial envelopes (MacDiamid J. A. et al Cancer Cell 11:431-445 (2007), electroporation that electrically opens muscle cell pores allowing more oloigonucleotide entry into the cell (Aihara, H. and Miyazaki, J., Nature Biotechnol. 16:867-870 (1998); Mir, L. M., et al., C R Acad. Sci. III 321:893-899 (1998), Mir, L. M., et al., Proc. Natl. Acad. Sci, USA 96:4262-4267 (1999); Mathiesen, I., Gene Ther. 6:508-514 (1999); Rizzuto, G., et al., Proc. Natl. Acad. Sci. USA 96:6417-6422 (1999); Schiffelers, R. M. et al Arthritis and Rheumatism 52:1314-1318 (2005); Golzio, M. et al Gene Ther. 12:246-251 (2005)); use of intravascular pressure or hydrodynamic delivery (Budker, V., et al., Gene Ther. 5:272-276 (1998); McCaffrey, A. P. et al Nature 418:28-39 (2002)).

In one embodiment, a siRNA molecule of the invention is designed or formulated to specifically target endothelial cells or tumor cells. For example, various formulations and conjugates can be utilized to specifically target endothelial cells or tumor cells, including PEI-PEG-folate, PEI-PEG-RGD, PEI-PEG-biotin, PEI-PEG-cholesterol, and other conjugates known in the art that enable specific targeting to endothelial cells and/or tumor cells.

In one embodiment, a compound, molecule, or composition for the treatment of ocular conditions (e.g., macular degeneration, diabetic retinopathy etc.) is administered to a subject intraocularly or by intraocular means. In another embodiment, a compound, molecule, or composition for the treatment of ocular conditions (e.g., macular degeneration, diabetic retinopathy etc.) is administered to a subject periocularly or by periocular means (see for example Ahlheim et al., International PCT publication No. WO 03/24420). In one embodiment, a siRNA molecule and/or formulation or composition thereof is administered to a subject intraocularly or by intraocular means. In another embodiment, a siRNA molecule and/or formulation or composition thereof is administered to a subject periocularly or by periocular means. Periocular administration generally provides a less invasive approach to administering siRNA molecules and formulation or composition thereof to a subject (see for example Ahlheim et al., International PCT publication No. WO 03/24420). The use of periocular administration also minimizes the risk of retinal detachment, allows for more frequent dosing or administration, provides a clinically relevant route of administration for macular degeneration and other optic conditions, and also provides the possibility of using reservoirs (e.g., implants, pumps or other devices) for drug delivery. In one embodiment, siRNA compounds and compositions of the invention are administered locally, e.g., via intraocular or periocular means, such as injection, iontophoresis (see, for example, WO 03/043689 and WO 03/030989), or implant, about every 1 50 weeks (e.g., about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks), alone or in combination with other compounds and/or therapy is herein. In one embodiment, siRNA compounds and compositions of the invention are administered systemically (e.g., via intravenous, subcutaneous, intramuscular, infusion, pump, implant etc.) about every 1 50 weeks (e.g., about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks), alone or in combination with other compounds and/or therapies described herein and/or otherwise known in the art.

In one embodiment, the nucleic acid molecules or the invention are administered to the CNS. Experiments have demonstrated the efficient in vivo uptake of nucleic acids by neurons. As an example of local administration of nucleic acids to nerve cells, Sommer et al., 1998, Antisense Nuc. Acid Drug Dev., 8, 75, describe a study in which a 15 mer phosphorothioate antisense nucleic acid molecule to c-fos is administered to rats via microinjection into the brain. Antisense molecules labeled with tetramethylrhodamine-isothiocyanate (TRITC) or fluorescein isothiocyanate (FITC) were taken up by exclusively by neurons thirty minutes post-injection. A diffuse cytoplasmic staining and nuclear staining was observed in these cells. As an example of systemic administration of nucleic acid to nerve cells, Epa et al., 2000, Antisense Nuc. Acid Drug Dev., 10, 469, describe an in vivo mouse study in which beta-cyclodextrin-adamantane-oligonucleotide conjugates were used to target the p75 neurotrophin receptor in neuronally differentiated PC12 cells. Following a two week course of IP administration, pronounced uptake of p75 neurotrophin receptor antisense was observed in dorsal root ganglion (DRG) cells. In addition, a marked and consistent down-regulation of p75 was observed in DRG neurons. Additional approaches to the targeting of nucleic acid to neurons are described in Broaddus et al., 1998, J. Neurosurg., 88(4), 734; Karle et al., 1997, Eur. J. Pharmocol., 340(2/3), 153; Bannai et al., 1998, Brain Research, 784(1,2), 304; Rajakumar et al., 1997, Synapse, 26(3), 199; Wu-pong et al., 1999, BioPharm, 12(1), 32; Bannai et al., 1998, Brain Res. Protoc., 3(1), 83; Simantov et al., 1996, Neuroscience, 74(1), 39. \Traditional approaches to CNS delivery that can be used include, but are not limited to, intrathecal and intracerebroventricular administration, implantation of catheters and pumps, direct injection or perfusion at the site of injury or lesion, injection into the brain arterial system, or by chemical or osmotic opening of the blood-brain barrier. Other approaches can include the use of various transport and carrier systems, for example though the use of conjugates and biodegradable polymers. Furthermore, gene therapy approaches, for example as described in Kaplitt et al., U.S. Pat. No. 6,180,613 and Davidson, WO 04/013280, can be used to express nucleic acid molecules in the CNS.

In one embodiment, the nucleic acid molecules or the invention are administered via pulmonary delivery, such as by inhalation of an aerosol or spray dried formulation administered by an inhalation device or nebulizer, providing rapid local uptake of the nucleic acid molecules into relevant pulmonary tissues. Solid particulate compositions containing respirable dry particles of micronized nucleic acid compositions can be prepared by grinding dried or lyophilized nucleic acid compositions, and then passing the micronized composition through, for example, a 400 mesh screen to break up or separate out large agglomerates. A solid particulate composition comprising the nucleic acid compositions of the invention can optionally contain a dispersant which serves to facilitate the formation of an aerosol as well as other therapeutic compounds. A suitable dispersant is lactose, which can be blended with the nucleic acid compound in any suitable ratio, such as a 1 to 1 ratio by weight. Aerosols of liquid particles comprising a nucleic acid composition of the invention can be produced by any suitable means, such as with a nebulizer (see for example U.S. Pat. No. 4,501,729). Nebulizers are commercially available devices which transform solutions or suspensions of an active ingredient into a therapeutic aerosol mist either by means of acceleration of a compressed gas, typically air or oxygen, through a narrow venturi orifice or by means of ultrasonic agitation. Suitable formulations for use in nebulizers comprise the active ingredient in a liquid carrier in an amount of up to 40% w/w preferably less than 20% w/w of the formulation. The carrier is typically water or a dilute aqueous alcoholic solution, preferably made isotonic with body fluids by the addition of, for example, sodium chloride or other suitable salts. Optional additives include preservatives if the formulation is not prepared sterile, for example, methyl hydroxybenzoate, anti-oxidants, flavorings, volatile oils, buffering agents and emulsifiers and other formulation surfactants. The aerosols of solid particles comprising the active composition and surfactant can likewise be produced with any solid particulate aerosol generator. Aerosol generators for administering solid particulate therapeutics to a subject produce particles which are respirable, as explained above, and generate a volume of aerosol containing a predetermined metered dose of a therapeutic composition at a rate suitable for human administration. One illustrative type of solid particulate aerosol generator is an insufflator. Suitable formulations for administration by insufflation include finely comminuted powders which can be delivered by means of an insufflator. In the insufflator, the powder, e.g., a metered dose thereof effective to carry out the treatments described herein, is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation. A second type of illustrative aerosol generator comprises a metered dose inhaler. Metered dose inhalers are pressurized aerosol dispensers, typically containing a suspension or solution formulation of the active ingredient in a liquified propellant. During use these devices discharge the formulation through a valve adapted to deliver a metered volume to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation can additionally contain one or more co-solvents, for example, ethanol, emulsifiers and other formulation surfactants, such as oleic acid or sorbitan trioleate, anti-oxidants and suitable flavoring agents. Other methods for pulmonary delivery are described in, for example U.S. Patent Application No. 20040037780, and U.S. Pat. Nos. 6,592,904; 6,582,728; 6,565,885.

In one embodiment, a siRNA molecule of the invention is administered iontophoretically, for example to a particular organ or compartment (e.g., the eye, back of the eye, heart, liver, kidney, bladder, prostate, tumor, CNS etc.). Non-limiting examples of iontophoretic delivery are described in, for example, WO 03/043689 and WO 03/030989, which are incorporated by reference in their entireties herein.

In one embodiment, a siRNA molecule of the invention is complexed with membrane disruptive agents such as those described in U.S. patent application Publication No. 20010007666, incorporated by reference herein in its entirety including the drawings. In another embodiment, the membrane disruptive agent or agents and the siRNA molecule are also complexed with a cationic lipid or helper lipid molecule, such as those lipids described in U.S. Pat. No. 6,235,310, incorporated by reference herein in its entirety including the drawings.

Thus, the invention features a pharmaceutical composition comprising one or more nucleic acid(s) of the invention in an acceptable carrier, such as a stabilizer, buffer, and the like. The oligonucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect. By “systemic administration” is meant in vivo systemic absorption or accumulation of drugs in the blood stream followed by distribution throughout the entire body. Administration routes that lead to systemic absorption include, without limitation: intravenous, subcutaneous, intraperitoneal, inhalation, oral, intrapulmonary and intramuscular. Each of these administration routes exposes the siRNA molecules of the invention to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells.

By “pharmaceutically acceptable formulation” is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85); biodegradable polymers, such as poly (DL-lactide-coglycolide) microspheres for sustained release delivery (Emerich, D F et al, 1999, Cell Transplant, 8, 47 58); and loaded nanoparticles, such as those made of polybutylcyanoacrylate. Other non-limiting examples of delivery strategies for the nucleic acid molecules of the instant invention include material described in Boado et al., 1998, J. Pharm. Sci., 87, 1308 1315; Tyler et al., 1999, FEBS Lett., 421, 280 284; Pardridge et al., 1995, PNAS USA., 92, 5592 5596; Boado, 1995, Adv. Drug Delivery Rev., 15, 73 107; Aldrian-Herrada et al., 1998, Nucleic Acids Res., 26, 4910 4916; and Tyler et al., 1999, PNAS USA., 96, 7053 7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601 2627; Ishiwata et al., Chem. Pharm. Bull. 1995, 43, 1005 1011). Such liposomes have been shown to accumulate selectively in tumors, presumably by extravasation and capture in the neovascularized target tissues (Lasic et al., Science 1995, 267, 1275 1276; Oku et al., 1995, Biochim. Biophys. Acta, 1238, 86 90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864 24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in a mixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanethiol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

In one embodiment, the invention comprises compositions suitable for administering nucleic acid molecules of the invention to specific cell types. For example, the asialoglycoprotein receptor (ASGPr) (Wu and Wu, 1987, J. Biol. Chem. 262, 4429 4432) is unique to hepatocytes and binds branched galactose-terminal glycoproteins, such as asialoorosomucoid (ASOR). In another example, the folate receptor is overexpressed in many cancer cells. Binding of such glycoproteins, synthetic glycoconjugates, or folates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611 620; Connolly et al., 1982, J. Biol. Chem., 257, 939 945). Lee and Lee, 1987, Glycoconjugate J., 4, 317 328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388 1395). The use of galactose, galactosamine, or folate based conjugates to transport exogenous compounds across cell membranes can provide a targeted delivery approach to, for example, the treatment of liver disease, cancers of the liver, or other cancers. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention. Non-limiting examples of such bioconjugates are described in Vargeese et al., U.S. Ser. No. 10/201,394, filed Aug. 13, 2001; and Matulic-Adamic et al., U.S. Ser. No. 10/151,116, filed May 17, 2002. In one embodiment, nucleic acid molecules of the invention are complexed with or covalently attached to nanoparticles, such as Hepatitis B virus S, M, or L evelope proteins (see for example Yamado et al., 2003, Nature Biotechnology, 21, 885). In one embodiment, nucleic acid molecules of the invention are delivered with specificity for human tumor cells, specifically non-apoptotic human tumor cells including for example T-cells, hepatocytes, breast carcinoma cells, ovarian carcinoma cells, melanoma cells, intestinal epithelial cells, prostate cells, testicular cells, non-small cell lung cancers, small cell lung cancers, etc.

Alternatively, certain siRNA molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591 5; Kashani-Sabet et al., 1992, Antisense Res. Dev., 2, 3 15; propulic et al., 1992, J. Virol., 66, 143241; Weerasinghe et al., 1991, J. Virol., 65, 55314; Ojwang et al., 1992, Proc. Natl. Acad. Sci. USA, 89, 10802 6; Chen et al., 1992, Nucleic Acids Res., 20, 4581 9; Sarver et al., 1990 Science, 247, 1222 1225; Thompson et al., 1995, Nucleic Acids Res., 23, 2259; Good et al., 1997, Gene Therapy, 4, 45. Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15 6; Taira et al., 1991, Nucleic Acids Res., 19, 5125 30; Ventura et al., 1993, Nucleic Acids Res., 21, 3249 55; Chowrira et al., 1994, J. Biol. Chem., 269, 25856.

In another aspect of the invention, RNA molecules of the present invention can be expressed from transcription units (see for example Couture et al., 1996, TIG., 12, 510) inserted into DNA or RNA vectors. The recombinant vectors can be DNA plasmids or viral vectors. siRNA expressing viral vectors can be constructed based on, but not limited to, adeno-associated virus, retrovirus, adenovirus, or alphavirus. In another embodiment, pol III based constructs are used to express nucleic acid molecules of the invention (see for example Thompson, U.S. Pas. Nos. 5,902,880 and 6,146,886). The recombinant vectors capable of expressing the siRNA molecules can be delivered as described above, and persist in target cells. Alternatively, viral vectors can be used that provide for transient expression of nucleic acid molecules. Such vectors can be repeatedly administered as necessary. Once expressed, the siRNA molecule interacts with the target mRNA and generates an RNAi response. Delivery of siRNA molecule expressing vectors can be systemic, such as by intravenous or intramuscular administration, by administration to target cells ex-planted from a subject followed by reintroduction into the subject, or by any other means that would allow for introduction into the desired target cell (for a review see Couture et al., 1996, TIG., 12, 510).

In one aspect the invention features an expression vector comprising a nucleic acid sequence encoding at least one siRNA molecule of the instant invention. The expression vector can encode one or both strands of a siRNA duplex, or a single self-complementary strand that self hybridizes into a siRNA duplex. The nucleic acid sequences encoding the siRNA molecules of the instant invention can be operably linked in a manner that allows expression of the siRNA molecule (see for example Paul et al., 2002, Nature Biotechnology, 19, 505; Miyagishi and Taira, 2002, Nature Biotechnology, 19, 497; Lee et al., 2002, Nature Biotechnology, 19, 500; and Novina et al, 2002, Nature Medicine, advance online publication doi: 10.103 8/nm725).

In another aspect, the invention features an expression vector comprising: a) a transcription initiation region (e.g., eukaryotic pol I, II or III initiation region); b) a transcription termination region (e.g., eukaryotic pol I, II or III termination region); and c) a nucleic acid sequence encoding at least one of the siRNA molecules of the instant invention, wherein said sequence is operably linked to said initiation region and said termination region in a manner that allows expression and/or delivery of the siRNA molecule. The vector can optionally include an open reading frame (ORF) for a protein operably linked on the 5′ side or the 3′-side of the sequence encoding the siRNA of the invention; and/or an intron (intervening sequences).

Transcription of the siRNA molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743 7; Gao and Huang 1993, Nucleic Acids Res., 21, 2867 72; Lieber et al, 1993, Methods Enzymol., 217, 47 66; Zhou et al., 1990, Mol. Cell. Biol., 10, 4529 37). Several investigators have demonstrated that nucleic acid molecules expressed from such promoters can function in mammalian cells (e.g. Kashani-Sabet et al., 1992, Antisense Res. Dev., 2, 3 15; Ojwang et al., 1992, Proc. Natl. Acad. Sci. USA, 89, 10802 6; Chen et al., 1992, Nucleic Acids Res., 20, 4581 9; Yu et al., 1993, Proc. Natl. Acad. Sci. USA, 90, 6340 4; L′Huillier et al., 1992, EMBO J, 11, 4411 8; Lisziewicz et al., 1993, Proc. Natl. Acad. Sci. U.S.A., 90, 8000 4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as siRNA in cells (Thompson et al., supra; Couture and Stinchcomb, 1996, supra; Noonberg et al, 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736. The above siRNA transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising a nucleic acid sequence encoding at least one of the siRNA molecules of the invention in a manner that allows expression of that siRNA molecule. The expression vector comprises in one embodiment; a) a transcription initiation region; b) a transcription termination region; and c) a nucleic acid sequence encoding at least one strand of the siRNA molecule, wherein the sequence is operably linked to the initiation region and the termination region in a manner that allows expression and/or delivery of the siRNA molecule.

In another embodiment the expression vector comprises: a) a transcription initiation region; b) a transcription termination region; c) an open reading frame; and d) a nucleic acid sequence encoding at least one strand of a siRNA molecule, wherein the sequence is operably linked to the 3′-end of the open reading frame and wherein the sequence is operably linked to the initiation region, the open reading frame and the termination region in a manner that allows expression and/or delivery of the siRNA molecule. In yet another embodiment, the expression vector comprises: a) a transcription initiation region; b) a transcription termination region; c) an intron; and d) a nucleic acid sequence encoding at least one siRNA molecule, wherein the sequence is operably linked to the initiation region, the intron and the termination region in a manner which allows expression and/or delivery of the nucleic acid molecule.

In another embodiment, the expression vector comprises: a) a transcription initiation region; b) a transcription termination region; c) an intron; d) an open reading frame; and e) a nucleic acid sequence encoding at least one strand of a siRNA molecule, wherein the sequence is operably linked to the 3′-end of the open reading frame and wherein the sequence is operably linked to the initiation region, the intron, the open reading frame and the termination region in a manner which allows expression and/or delivery of the siRNA molecule.

### V. CERTAIN KITS

The instant teachings also provide kits designed to facilitate the subject methods. Kits serve to expedite the performance of the disclosed methods by assembling two or more components required for carrying out certain methods. Kits can contain components in pre-measured unit amounts to minimize the need for measurements by end-users and can also include instructions for performing one or more of the disclosed methods. Typically, kit components are optimized to operate in conjunction with one another. The disclosed kits may be used to identify, detect, and/or quantitate target oligonucleotides, including small RNA molecules and oligonucleotides comprising deoxyribonucleotides. In certain embodiments, kits comprising a reverse transcription primer comprises an oligonucleotide molecule-binding portion having an oligonucleotide recognition sequence comprising at least 2 nucleotides at the 3′ region that are complementary to a region of the oligonucleotide molecule and an extension tail comprising at least 2 nucleotides at the 5′ region; a forward primer, wherein the forward primer comprises an oligonucleotide molecule-binding portion comprising at least 2 nucleotides that are the same as a region of the oligonucleotide molecule; and a reverse primer. In certain embodiments, such kits comprise a first primer set that includes a forward and a corresponding reverse primer. In certain embodiments, the disclosed kits further comprise, a second primer set, including without limitation a universal forward primer, a universal reverse primer, or both; a reporter probe; a reporter group; a reaction vessel, including without limitation, a multi-well plate or a microfluidic device; a substrate; a buffer or buffer salt; a surfactant; or combinations thereof. In certain embodiments, the disclosed kits may further comprise a first extending enzyme, a second extending enzyme, and/or a third extending enzyme.

### EXAMPLES

### Reagents

The following DNA oligos were used: Reverse Transcription (RT-) primer: 5′-GTATCC AGT GCA GGG TCC GGT CGA-3′ (SEQ ID NO: 1); Forward (FW-) primer: 5′-GCG TTG AGG TTT GAA ATC-3′ (SEQ ID NO: 2); Reverse (Rev-) primer: 5′-GTA TCC AGT GCA GGG TCC-3′ (SEQ ID NO: 3). siRNA anti-sense sequence against VEGFR2: 5′-UUG AGG UUU GAA AUC GAC Cx-3′ (SEQ ID NO: 4) (x is a C3-linker).

TaqMan MicroRNA Reverse transcription kit (Part no. 4346906), Taqman 2x Universal PCR Master Mix (Part no. 4324018) and MicroAmp Fast optical 96-well reaction plates (Part no. 4366597) were purchased form Applied Biosystems. SYBR Green I (S7563) was obtained from Invitrogen.

### Protocol for Two-Step RT-PCR:

Plasma was diluted 10, 100 and 1000 times, respectively in RNAse-free water (Optimal dilutions were established empirically). Standard curves were obtained by serial dilutions of double strand siRNA in RNAse-free H₂O. In the first step, samples were heated for 5 minutes at 95° C. and allowed to cool down to RT on the bench. Subsequently, 3 μl sample was mixed with 12 μl RT-buffer containing: 0.15 μl 100 mM dNTPs, 2 μl 0.5 μM RT-primer, 1.5 μl 10x RT-buffer, 1.0 μl Multiscribe reverse transcriptase 50U/μl 0.19 μl RNAse inhibitor 20 U/μl and 7.16 μl RNAse-free H₂O. The RT-mix was applied to a 96 well MicroAmp plate and incubated at 16° C. (30 minutes), 42° C. (30 minutes), 85° C. (5 minutes) and then held at 4° C. In the second step, 3 μl of the RT-reaction was mixed with 12 μl PCR buffer containing: 0.3 μl 10 μM FW-primer, 0.3 μl 10 μM Universal Rev-primer, 3.8 μl RNAse-free H2O, 7.5 μl Taqman 2x Universal PCR Master Mix and 0.1 μl 100x SYBR Green 1. The PCR reaction was performed with the following parameters: 1 cycle: 10 minutes 95° C.; 45 cycles: 15 seconds 95° C., 1 minute 50° C.

### Data Analysis:

The data was analyzed using the system software (7500 or 7900HT Fast System software). For each sample the deltaCt value (Taqman threshold cycle) was calculated by substracting the Ct-value of the no template control sample. The deltaCt value was converted to a linear signal with the formula: EXP(-In(2)*deltaCt). Standard curves were plotted in EXCEL. Depicted values represent the siRNA concentration per microliter plasma (average signal of the three dilutions with their corresponding standard deviation).

The results of the experiments are depicted in FIG. 1 which shows the quantification of siRNAs in plasma using two-step RT-PCR. Mice (three animals per group) were treated inter peritoneal (i.p) or by gavage (p.o: per os) with either 10 mg/kg siRNA or 100 mg/kg. Plasma was obtained within minutes (TO) after administration. The Upper panel shows the Standard curve of modified siRNAs directed against VEGFR2. Amount of siRNA was plotted against signal intensity. The slope (0.9882), intercept (0.9564) and R-squared (0.9984) were calculated using linear regression. The Lower panel shows the bar graphs represents the amount of VEGFR2-siRNA (fmol) per μl plasma detected by RT-PCR in one mouse of each group. For comparison, plasma (1.25 μl) of each animal was loaded on a gel and stained with SYBR GOLD. As a reference, a dilution series ranging from 0.1 to 6.3 pmol siRNA was also included on the gel.

### Example 2

### Detection of siRNAs in Plasma Using One-Step RT-PCR

### Reagents:

For the detection of the siRNA anti-sense sequence against VEGFR2: 5′-UUG AGG UUU GAA AUC GAC Cx-3′ (SEQ ID NO: 5) (x is a C3-linker), the following DNA oligos were used: Reverse Transcription (RT-)primer: 5′ GTA TCC AGT GCA GGG TCC GGT CGA-3′(SEQ ID NO: 6); Forward (FW-) primer: 5′-GCG TTG AGG TTT GAA ATC-3′(SEQ ID NO: 7): Reverse (Rev-) primer: 5′-GTA TCC AGT GCA GGG TCC-3′(SEQ ID NO: 8).

TaqMan MicroRNA Reverse transcription kit (Part no. 4346906) and MicroAmp Fast optical 96-well reaction plates (Part no. 4366597) were purchased form Applied Biosystems. SYBR Green I (S7563) and ROX Reference dye (cat.no: 12223-012) were obtained from Invitrogen. Taq polymerase (cat. No: 04 738 225 001) was obtained from Roche.

### Sample Description:

Tumours were grown for 7 days. On Day 7 plasma was collected from naïve mice to be treated with either vehicle (mouse 1 to 6), 0.2 mg VEGFR2 siRNA (mouse 7 to 12) or 2.0 mg VEGFR2 siRNA (mouse 13 to 18). Subsequently, plasma was isolated 1 hour after administration of the VEGFR2 siRNA (mouse 19 to 24: 0.2 mg VEGFR2 siRNA/mouse 25 to 30: 2.0 mg VEGFR2 siRNA). On day 14, plasma was isolated from the vehicle treated mice (31 to 36); 0.2 mg VEGFR2 siRNA treated mice (37 to 42) and the 2.0 mg VEGFR2 treated animals (43-48). This data set reflects the siRNA level after 24 hours post siRNA administration. Plasma was also collected 1 hour after siRNA treatment (mouse 49 to 54: 0.2 mg VEGFR2 siRNA/mouse 55 to 60: 2.0 mg VEGFR2 siRNA).

### Protocol for One-Step RT-PCR:

Plasma was diluted 10 times in sterile RNAse free water. VEGFR2 siRNA standard was prepared and both sample and standards were heated for 5 minutes at 95° C. and subsequently chilled on ice. 5 μl of sample was mixed with 10 μl RT-PCR buffer containing: 0.15 μl 100 mM dNTPs, 0.1 μl 10 μM RT-primer, 0.3 μl 10 μM FW-primer, 0.3 μl 10 μM Rev-primer, 1.5 μl 10x RT-buffer, 0.1 μl 100x SYBR Green I, 0.03 μl ROX Reference Dye, 0.5 μl Multiscribe reverse transcriptase (50 U/μl), 0.19 μl RNAse inhibitor (20 U/μl), 0.15 μl Taq polymerase (1 U/μl) and 6.68 μl RNAse-free H₂O. The PCR mix was applied to a 96 well MicroAmp plate and subsequently incubated at 16° C. (30 minutes), 42° C. (30 minutes), 95° C. (10 minutes) followed by 45 cycles of 95° C. (15 seconds), 50° C. (1 minute, data acquisition) using the 9800 Fast Thermal Cycler (Applied Biosystems). Data was analyzed using the system software (7500 Fast System software).

### Data Analysis:

Standard curve was plotted in EXCEL and the values for a and b in the formula y=ax+b were obtained using linear regression. Ct values of the samples were converted to femtogram siRNA per μl plasma using this formula. Depicted values represent the average of all the animals within the same group with the corresponding standard deviation.

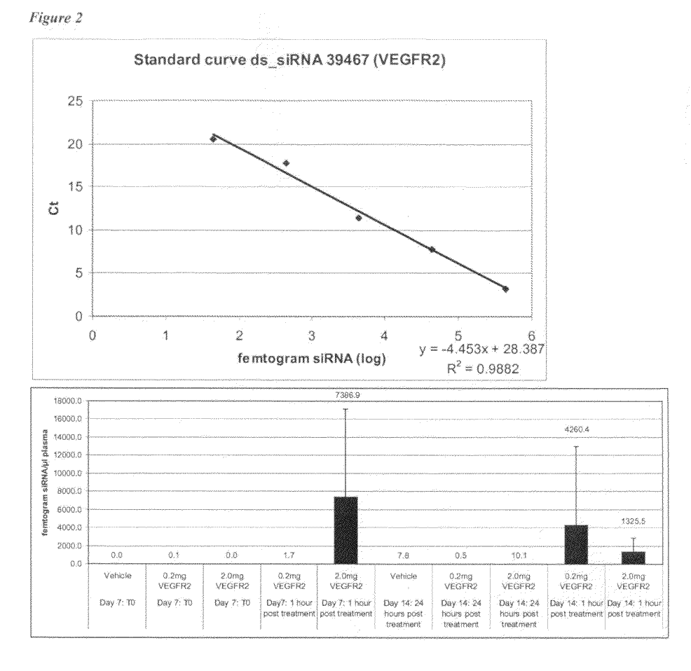

FIG. 2 shows the quantification of siRNAs in plasma using one-step RT-PCR. Mice were treated by gavage (p.o: per os) with either vehicle or vehicle containing 0.2 mg or 2.0 mg siRNA directed against the mRNA encoding VEGFR2. Plasma was isolated from naïve animals (Day 7:T0), one hour after treatment (Day 7: 1 hour post treatment and Day 14: 1 hour post treatment) and 24 hours after the last treatment (Day 14: 24 hours post treatment). siRNA. The amount of siRNA detected within plasma was calculated using the standard curve (upper panel). The bargraph represents the average siRNA levels within each group with its corresponding standard deviation (lower panel).

### Example 3

### Detection of siRNAs in Tissues Using Two-Step RT-PCR

Comparing SYBR Green I Based Detection with FAM/TAMRA Probes:

### Reagents:

For the SYBR Green I based detection of the siRNA, the following DNA oligos were used: Reverse Transcription (RT-) primer: 5′-GCG TAT CGA GTG CAG GAT CCA CTT TC-3′(SEQ ID NO:9); Forward (FW-) primer: 5′-GCG TGT TCT TGT CAT TGA-3′(SEQ ID NO:10); Reverse (Rev-) primer: 5′-GCG TAT CGA GTG CAG G-3′(SEQ ID NO:11). For the FMA/TAMRA based detection of the siRNA, the following DNA oligos were used: Reverse Transcription (RT-) primer: 5′-GCG TAT CGA GTG CAG GAT CCT GGA AGC AGC AAC TTT C-3′(SEQ ID NO:12); Forward (FW-) primer: 5′-GCG TGT TCT TGT CAT TGA-3′ (SEQ ID NO:13); Reverse (Rev-) primer: 5′-GCG TAT CGA GTG CAG G-3′(SEQ ID NO:14); probe: 5′FAM-TOG AAG CAG CAA CTT TCA ATG A-3′TAMRA (SEQ ID NO:15). Anti-sense siRNA sequence ND9227: 5′-UGU UCU UGU cAU UGA AAG UTsT-3′(SEQ ID NO:16). Anti-sense siRNA sequence AD1955: 5′-UCGAAGuACUcAGCGuAAGTsT-3′ (SEQ ID NO:17). TaqMan MicroRNA Reverse transcription kit (Part no. 4346906) and MicroAmp Fast optical 96-well reaction plates (Part no. 4366597) were purchased form Applied Biosystems. ROX Reference dye (cat.no: 12223-012) was obtained from Invitrogen and Taq polymerase (cat.no: 11 647 679 001) was obtained from Roche.

### Sample Description:

Rats 41 and 42: hypotonic saline (100 mOsmol/kg water). Rats 49 and 50: 10 mg/kg siRNA AD1955, 2 doses at 24 hr intervals and harvested at 24 hours after final treatment. Rats 57 and 58: 50 mg/kg siRNA AD1955, 2 doses at 24 hr intervals and harvested at 24 hours after final treatment. Rats 65 and 66: 10 mg/kg siRNA ND9227, 2 doses at 24 hr intervals and harvested at 24 hours after final treatment. Rats 73 and 74: 50 mg/kg siRNA ND9227, 2 doses at 24 hr intervals and harvested at 24 hours after final treatment.

sirna Isolation from Tissues:

Pulverized frozen lung tissues were homogenized in TRIZOL (1 ml TRIZOL per 100 mg tissue) using a hand-held Polytron (PT1200, Kinematica AG, Switzerland). Homogenates were incubated for 5 minutes at room temperature. After addition of Chloroform (200 μl/1 ml TRIZOL), samples were vigorously shaken for 15 seconds followed by 15 minutes centrifugation at 12000 rpm (2-8° C.). Upper phase was transferred to a fresh tube and RNA was precipitated by adding 2-Propanol (500 μl/1 ml TRIZOL) followed by a 10 minutes incubation at room temperature. After 10 minutes centrifugation at 12000 rpm (2-8° C.), the pellet was washed with 1 ml ice cold 75% EtOH and resuspended in RNAse-free H₂O. RNA concentrations were determined using the NanoDrop ND-100 Spectrophotometer (Witec AG). For RT-PCR purpose, all samples were adjusted to a final RNA concentration of 10 ng/μl.

### Protocol for Two-Step RT-PCR:

RNA samples (10 ng/μl) were heated for 5 minutes at 95° C. and chilled on ice. 5 μl sample was mixed with 10 μl RT-buffer containing: 0.15 μl 100 mM dNTPs, 0.1 μl 10 μM RT-primer, 1.5 μl 10x RT-buffer, 0.5 μl Multiscribe reverse transcriptase 50 U/μl 0.19 μl RNAse inhibitor 20 U/μl and 7.56 μl RNAse-free H₂O. The RT-mix was applied to a 96 well MicroAmp plate and incubated at 16° C. (20 minutes), 42° C. (20 minutes), 85° C. (5 minutes) and then held at 4° C. Subsequently, 5 μl of the RT-reaction was mixed with 10 μl PCR buffer containing: 0.15 μl 100 mM dNTPs; 0.3 μl 10 μM FW-primer; 0.3 μl 10 μM Rev-primer; 0.3 μl 30 μM FAM/TAMRA probe; 1.5 μl 10x PCR-buffer(+MgCl2); 0.03 μl ROX Reference Dye; 0.12 μl Taq polymerase (5 U/μl) and 7.3 μl RNAse-free H₂O. With SYBR Green I based detection, the FAM/TAMRA probe was substituted with 0.1 μl 100x CYBR Green I and the final volume was adjusted accordingly. The PCR mix was applied to a 96 well MicroAmp plate and subsequently incubated at 95° C. (5 minutes) followed by 40 cycles of 95° C. (15 seconds), 58° C. (30 seconds) and 72° C. (1 minute, data acquisition) using the 9800 Fast Thermal Cycler (Applied Biosystems). Data was analyzed using the system software (7500 Fast System software).

### Data Analysis:

For each sample the deltaCt value (Taqman threshold cycle) was calculated by substracting the Ct-value of the no template control sample. The deltaCt value was converted to relative signal intensity with the formula: EXP(-In(2)*deltaCt). Depicted values represent the average signal of two independent RT-PCR reactions and their standard deviation.

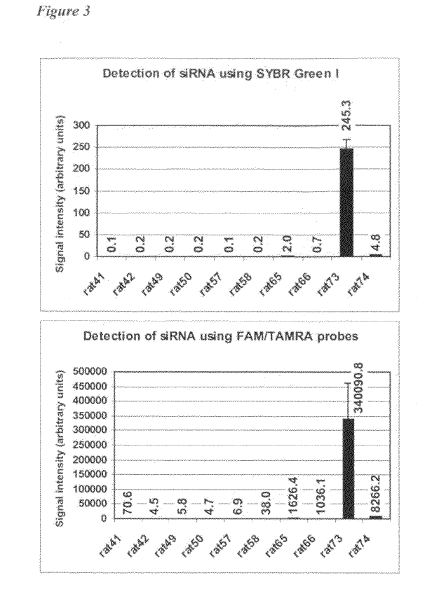

FIG. 3 shows the comparison of two-step RT-PCR based detection of siRNAs ND9227 using SYBR Green I or FAM/TAMRA labeled probes as readout. Two-step RT-PCR was performed on 50 ng total RNA obtained from rat lungs treated with either siRNA ND-9227 (10 mg/kg; rats 65/66, 50 mg/kg; rats 73-74) or AD1955 (10 mg/kg; rats 49/50, 50 mg/kg; rats 57-58) or left untreated (rats 41/42). Relative expression, reflected by the signal intensity, was established using either SYBR Green I (upper panel) as read out for signal intensity or a FAM/TAMRA labeled probe (lower panel). Bar graphs represent the average of two independent RT-PCR reactions with their corresponding standard deviation (n=2,±stdev).

### Example 4

### Quantitative Detection of siRNA in Rat Lung Using FAM/TAMRA Probes

### Reagents:

The following DNA oligos were used: Reverse Transcription (RT-) primer: 5′-GCG TAT CGA GTG CAG GAT CCT GGA AGC AGC AAC TTT C-3′(SEQ ID NO:18); Forward (FW-) primer: 5′-GCG TGT TCT TGT CAT TGA-3′(SEQ ID NO:19); Reverse (Rev-) primer: 5′-GCG TAT CGA GTG CAG G-3′(SEQ ID NO:20); probe: 5′FAM-TGG AAG CAG CAA CTT TCA ATG A-3′TAMRA (SEQ ID NO:21). Anti-sense siRNA sequence ND9227: 5′-UGU UCU UGU cAU UGA AAG UTsT-3′ (SEQ ID NO:22). TaqMan MicroRNA Reverse transcription kit (Part no. 4346906) and MicroAmp Fast optical 96-well reaction plates (Part no. 4366597) were purchased form Applied Biosystems. ROX Reference dye (cat.no: 12223-012) was obtained from Invitrogen and Taq polymerase (cat.no: 11 647 679 001) was obtained from Roche.

### Sample Description:

Rats 41 and 42: hypotonic saline (100 mOsmol/kg water). Rats 49 and 50: 10 mg/kg siRNA AD1955, 2 doses at 24 hr intervals and harvested at 24 hours after final treatment. Rats 57 and 58: 50 mg/kg siRNA AD1955, 2 doses at 24 hr intervals and harvested at 24 hours after final treatment. Rats 65 and 66: 10 mg/kg siRNA ND9227, 2 doses at 24 hr intervals and harvested at 24 hours after final treatment. Rats 73 and 74: 50 mg/kg siRNA ND9227, 2 doses at 24 hr intervals and harvested at 24 hours after final treatment.

siRNA Isolation from Tissues:

Pulverized frozen lung tissues were homogenized in TRIZOL (1 ml TRIZOL per 100 mg tissue) using a hand-held Polytron (PT1200, Kinematica AG, Switzerland). Homogenates were incubated for 5 minutes at room temperature. After addition of Chloroform (200 μl/1 ml TRIZOL), samples were vigorously shaken for 15 seconds followed by 15 minutes centrifugation at 12000 rpm (2-8° C.). Upper phase was transferred to a fresh tube and RNA was precipitated by adding 2-Propanol (500 μl/1 ml TRIZOL) followed by a 10 minutes incubation at room temperature. After 10 minutes centrifugation at 12000 rpm (2-8° C.), the pellet was washed with 1 ml ice cold 75% EtOH and resuspended in RNAse-free H₂O. RNA concentrations were determined using the NanoDrop ND-100 Spectrophotometer (Witec AG). For RT-PCR purpose, all samples were adjusted to a final RNA concentration of 10 ng/μl.

### Protocol for Two-Step RT-PCR:

RNA samples (10 ng/μl) were heated for 5 minutes at 95° C. and chilled on ice. 5 μl sample was mixed with 10 μl RT-buffer containing: 0.15 μl 100 μM dNTPs, 0.1 μl 10 μM RT-primer, 1.5 μl 10x RT-buffer, 0.5 μl Multiscribe reverse transcriptase 50 U/μl, 0.19 μl RNAse inhibitor 20 U/μl and 7.56 μl RNAse-free H₂O. The RT-mix was applied to a 96 well MicroAmp plate and incubated at 16° C. (20 minutes), 42° C. (20 minutes), 85° C. (5 minutes) and then held at 4° C. Subsequently, 5 μl of the RT-reaction was mixed with 10 μl PCR buffer containing: 0.15 μl 100 mM dNTPs; 0.3 μl 10 μM FW-primer; 0.3 μl 10 μM Rev-primer; 0.3 μl 30 μM FAM/TAMRA probe; 1.5 μl 10x PCR-buffer(+MgCl2); 0.0411 ROX Reference Dye; 0.12 μl Taq polymerase (5 U/μl) and 7.3 μl RNAse-free H₂O. The PCR mix was applied to a 96 well MicroAmp plate and subsequently incubated at 95° C. (5 minutes) followed by 40 cycles of 95° C. (15 seconds) and 60° C. (1 minute, data acquisition) using the 9800 Fast Thermal Cycler (Applied Biosystems). Data was analyzed using the system software (7500 Fast System software).

### Data Analysis:

For each sample the deltaCt value (Taqman threshold cycle) was calculated by substracting the Ct-value of the no template control sample. The deltaCt value was converted to relative signal intensity with the formula: EXP(-In(2)*deltaCt). Amounts of siRNA in the sample were calculated using the standard curve. Depicted values represent the average signal of three independent RT-PCR reactions and their standard deviation.

FIG. 4 shows the results from the absolute quantification of siRNA in rat lung. Two-step RT-PCR performed on serial dilutions of siRNA ND9227 (upper panel) and 50 ng total RNA obtained from rat lungs (lower panel) treated with either siRNA ND-9227 (10 mg/kg; rats 65/66, 50 mg/kg; rats 73-74) or AD1955 (10 mg/kg; rats 49/50, 50 mg/kg; rats 57-58) or left untreated (rats 41/42). Bar graphs represents the average siRNA concentration of three independent RT-PCR reactions with their corresponding standard deviation (n=3, ±stdev).

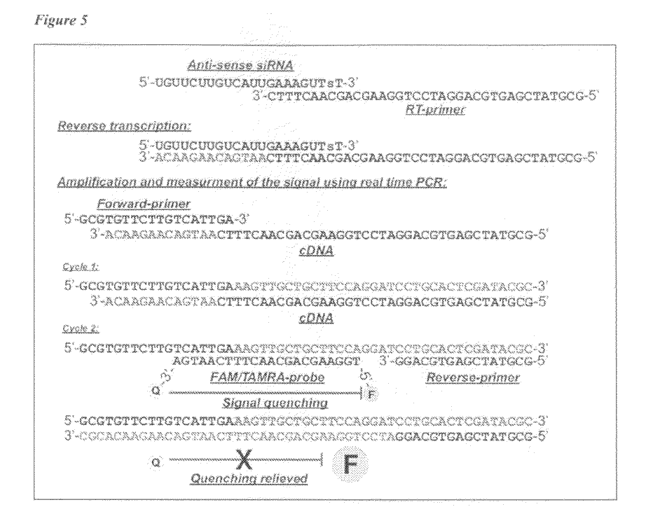

FIG. 5 depicts the outline of siRNA detection using FAM/TAMRA probes. During reverse transcription, anti-sense RNA is recognized by the Reverse-Transcription (RT)-primer and single strand copy DNA (cDNA) is generated. Subsequently, in the first cycle of the polymerase chain reaction (PCR), the cDNA serves as template for the forward primer to generate double strand DNA molecule. In the second PCR cycle, this newly synthesized DNA strand serves as docking site for the FAM/TAMRA probe and the reverse-primer. When no template is present, the probe is intact and the proximity of the reporter dye to the quencher dye results in suppression of the reporter fluorescence. However, when the probe binds to its target sequence, the 5′-3″ nuclease activity of the DNA polymerase system cleaves the probe between the reporter and the quencher resulting in detection of a signal. During this process, the probe fragments are displaced from the target, and polymerization of the strand continues. The 3′ end of the probe is blocked to prevent extension of the probe during PCR. This process occurs in every cycle and does not interfere with the exponential accumulation of product.

### REFERENCES

1. Fire, Andrew; Xu, SiQun; Montgomery, Mary K.; Kostas, Steven A.; Driver, Samuel E.; Mello, Craig C. Potent and specific genetic interference by double-stranded RNA in Caenorhabditis elegans. Nature (1998) 391: 806-811.
2. Elbashir, Sayda M.; Harborth, Jens; Lendeckel, Winfried; Yalcin, Abdullah; Weber, Klaus; Tuschl, Thomas. Duplexes of 21-nucleotide RNAs mediate RNA interference in cultured mammalian cells. Nature (2001) 411: 494-498.
3. Meister, Gunter; Tuschl, Thomas. Mechanisms of gene silencing by double-stranded RNA. Nature (2004) 431: 343-349.
4. Filipowicz, Witold. RNAi: The nuts and bolts of the RISC machine. Cell (2005) 122: 17-20.
5. Mukherji, Mridul; Bell, Russell; Supekova, Lubica; Wang, Yan; Orth, Anthony P.; Batalov, Serge; Miraglia, Loren; Huesken, Dieter; Lange, Joerg; Martin, Christopher; Sahasrabudhe, Sudhir; Reinhardt, Mischa; Natt, Francois; Hall, Jonathan; Mickanin, Craig; Labow, Mark; Chanda, Sumit K.; Cho, Charles Y.; Schultz, Peter G. Genome-wide functional analysis of human cell-cycle regulators. Proceedings of the National Academy of Sciences of the United States of America (2006) 103: 14819-14824.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, or related fields are intended to be within the scope of the following claims.

1. A method for improved sensitivity of identifying or quantifying an oligonucleotide molecule in a sample, the method comprising the steps of:
hybridizing a reverse transcription primer to the oligonucleotide molecule, wherein the reverse transcription primer comprises an oligonucleotide molecule-binding portion having an oligonucleotide recognition sequence comprising at least 2 nucleotides at the 3′ region that are complementary to a region of the oligonucleotide molecule and an extension tail comprising at least 2 nucleotides at the 5′ region;
extending the hybridized reverse transcription primer with a first extending enzyme to generate a reverse-transcribed product;
hybridizing a forward primer to the reverse-transcribed product, wherein the forward primer comprises an oligonucleotide molecule-binding portion comprising at least 2 nucleotides that are the same as a region of the oligonucleotide molecule;
extending the hybridized forward primer with a second extending enzyme to generate a first amplicon;
hybridizing a reverse primer to the first amplicon;
extending the hybridized reverse primer with the second extending enzyme to generate a second amplicon complementary to the first amplicon; detecting the amplification product;
and thereby identifying or quantifying the oligonucleotide molecule.

2. The method of claim 1, wherein the oligonucleotide molecule is selected from the group consisting of: a small RNA molecule, a DNA molecule, a modified RNA molecule, a modified DNA molecule, an aptamer, a ribozyme, a decoy oligonucleotide, and an immunostimulatory oligonucleotide.

3. The method of claim 1, wherein the oligonucleotide has a length comprising 10-30 nucleotides.

4. The method of claim 1, wherein the oligonucleotide is chemically modified.

5. The method of claim 1, wherein the oligonucleotide is double stranded.

6. The method of claim 1, where the oligonucleotide is an siRNA.

7. The method of claim 1, wherein the reverse transcriptase primer further comprises a reverse primer sequence.

8. The method of claim 1, wherein the reverse transcriptase primer further comprises a probe sequence.

9. The method of claim 8, wherein the probe sequence is positioned at a position selected from the group consisting of between the forward and reverse primer, or within the reverse transcriptase primer.

10. The method of claim 1, wherein the first primer and the second primer are unmodified primers.

11. The method of claim 1, wherein the first primer and the second primer are modified primers.

12. The method of claim 11, wherein the first or second modified primer is modified with a modification comprising one or more of the following: an LNA residue, peptide nucleic acid residue, 2′-modified RNA residue, a modified nucleobase and a combination thereof.

13. The method of claim 1, wherein the hybridization occurs in a single reaction mixture comprising the reverse transcriptase primer, the reverse primer and the forward primer.

14. The method of claim 1, wherein the hybridization occurs in two separate reaction mixtures, wherein the reverse transcriptase primer is present in a first reaction mixture and is used to generate a reverse transcribed product, and the forward and reverse primers are present in a second reaction mixture, wherein the reverse transcribed product from the first reaction mixture is used as a template for the forward and reverse primers in the second reaction mixture.

15. The method of claim 1, wherein the oligonucleotide molecule-binding portion of the reverse transcriptase primer comprises a nucleotide sequence that is at least 90% complementary with the oligonucleotide molecule.

16. The method of claim 15, wherein the oligonucleotide molecule-binding portion of the reverse transcriptase primer comprises about 2-17 nucleotides that are complementary with the oligonucleotide molecule, and wherein the oligonucleotide molecule is about 4-19 nucleotides in length.

17. The method of claim 1, wherein the oligonucleotide molecule-binding portion of the reverse primer comprises about 2-30 nucleotides that are complementary to the region of the oligonucleotide molecule.

18. The method of claim 1, wherein the oligonucleotide molecule-binding portion of the forward primer comprises about 2-30 nucleotides having the same sequence as the region of the oligonucleotide molecule.

19. The method of claim 1, wherein the step of detecting the amplification product comprises detecting the first amplicon with a first detection probe, detecting the second amplicon with a second detection probe, and detecting both the first and second amplicons with multiple detection probes.

20. The method of claim 19, wherein the first detection probe is a double stranded DNA intercalating agent.

21. The method of claim 19, wherein the first detection probe is SYBR Green.

22. The method of claim 19, wherein the first and/or the second detection probe is a signal emitting probe that binds with the oligonucleotide molecule binding portion using Watson-Crick base pairing.

23. The method of claim 22, wherein the signal emitting probe is selected from the group consisting of a FAM, VIC, JOE, NED, CY5 dye, CY3-dye, TAMRA labeled probe, an MBG probe, a scorpion probe and a molecular beacon.

24. The method of claim 23, wherein the detection probe comprises a FAM/TAMRA detection group.

25. The method of claim 1, wherein the sensitivity for quantifying the oligonucleotide molecule is improved by a factor of at least 10-100,000 fold detected using a signal intensity readout.

26. The method of claim 1, wherein the sensitivity for quantifying the oligonucleotide is improved by a factor of at least 100-10,000 fold detected using a signal intensity readout.

27. The method of claim 1, wherein the sensitivity for quantifying the oligonucleotide is improved to detect oligonucleotide molecules in a concentration range of about 1 molecule to about 1×10¹⁰ molecules.

28. The method of claim 1, wherein the sensitivity for quantifying the oligonucleotide is improved to detect oligonucleotide molecules in a concentration range of about 100 molecules to about 1×10⁹ molecules.

29. The method of claim 1, wherein the oligonucleotide molecule is detected after administration of the oligonucleotide molecule into a subject by a clinically relevant route selected but from the group consisting of: intratracheal, intranasal, intracerebral, intrathecal, colorectal, oral, intramuscular, intraarticular, topical including vaginal, lung delivery, intraocular, intraperitoneal, intravenous, and subcutaneous administration.

30. The method of claim 1, wherein the oligonucleotide molecule is formulated with a pharmaceutical carrier capable of facilitating delivery to and/or uptake by the target cells, wherein the carrier comprises a composition selected from, the group consisting of: a neutral liposome, a cationic liposome or lipoplex, a cationic polymer or polyplex, a neutral polymer, a nanoparticle, a double stranded RNA binding protein, calcium phosphate, a cell penetrating peptide, a viral protein, a viral particle, an antibody and an empty bacterial envelope.

31. The method of claim 1, wherein the sample is selected from the group consisting of a fluid, a tissue, a cell, and a tumor.


SEQUENCE LISTING (22 sequences)

SEQ ID NO: 1 - DNA, 24 bp, Artificial sequence
  Synthetic oligonucleotide
gtatccagtgcagggtccggtcga

SEQ ID NO: 2 - DNA, 18 bp, Artificial sequence
  Synthetic oligonucleotide
gcgttgaggtttgaaatc

SEQ ID NO: 3 - DNA, 18 bp, Artificial sequence
  Synthetic oligonucleotide
gtatccagtgcagggtcc

SEQ ID NO: 4 - RNA, 20 nt, Artificial sequence
  Synthetic oligonucleotide
uugagguuugaaaucgaccn

SEQ ID NO: 5 - RNA, 20 nt, Artificial sequence
  Synthetic oligonucleotide
uugagguuugaaaucgaccn

SEQ ID NO: 6 - DNA, 24 bp, Artificial sequence
  Synthetic oligonucleotide
gtatccagtgcagggtccggtcga

SEQ ID NO: 7 - DNA, 18 bp, Artificial sequence
  Synthetic oligonucleotide
gcgttgaggtttgaaatc

SEQ ID NO: 8 - DNA, 18 bp, Artificial sequence
  Synthetic oligonucleotide
gtatccagtgcagggtcc

SEQ ID NO: 9 - DNA, 26 bp, Artificial sequence
  Synthetic oligonucleotide
gcgtatcgagtgcaggatccactttc

SEQ ID NO: 10 - DNA, 18 bp, Artificial sequence
  Synthetic oligonucleotide
gcgtgttcttgtcattga

SEQ ID NO: 11 - DNA, 16 bp, Artificial sequence
  Synthetic oligonucleotide
gcgtatcgagtgcagg

SEQ ID NO: 12 - DNA, 37 bp, Artificial sequence
  Synthetic oligonucleotide
gcgtatcgagtgcaggatcctggaagcagcaactttc

SEQ ID NO: 13 - DNA, 18 bp, Artificial sequence
  Synthetic oligonucleotide
gcgtgttcttgtcattga

SEQ ID NO: 14 - DNA, 16 bp, Artificial sequence
  Synthetic oligonucleotide
gcgtatcgagtgcagg

SEQ ID NO: 15 - DNA, 22 bp, Artificial sequence
  Synthetic oligonucleotide
tggaagcagcaactttcaatga

SEQ ID NO: 16 - DNA, 22 bp, Artificial sequence
  Synthetic oligonucleotide
uguucuugucauugaaagutst

SEQ ID NO: 17 - DNA, 22 bp, Artificial sequence
  Synthetic oligonucleotide
ucgaaguacucagcguaagtst

SEQ ID NO: 18 - DNA, 36 bp, Artificial sequence
  Synthetic oligonucleotide
gcgtatcgagtgcaggatcctggaagcagcaacttt

SEQ ID NO: 19 - DNA, 18 bp, Artificial sequence
  Synthetic oligonucleotide
gcgtgttcttgtcattga

SEQ ID NO: 20 - DNA, 16 bp, Artificial sequence
  Synthetic oligonucleotide
gcgtatcgagtgcagg

SEQ ID NO: 21 - DNA, 22 bp, Artificial sequence
  Synthetic oligonucleotide
tggaagcagcaactttcaatga

SEQ ID NO: 22 - DNA, 22 bp, Artificial sequence
  Synthetic oligonucleotide
uguucuugucauugaaagutst